(19) United States
(12) Patent Application Publication
(10) Pub. No.: US 2025/0302960 A1
(43) Pub. Date: Oct. 2, 2025

(54) IMMUNE CELL WITH DOWN-REGULATED CELL ADHESION CAPABILITY AND MEDICAL USE THEREOF

(71) Applicant: SUZHOU IMMUNOHEAD BIOTECHNOLOGY CO., LTD., Suzhou, Jiangsu (CN)

(72) Inventors: Shuangshuang ZHANG, Suzhou, Jiangsu (CN); Ruidong HAO, Suzhou, Jiangsu (CN); Jinhui HU, Suzhou, Jiangsu (CN); Kaixuan XING, Suzhou, Jiangsu (CN); Qingshan SHEN, Suzhou, Jiangsu (CN)

(21) Appl. No.: 18/844,888

(22) PCT Filed: Mar. 9, 2023
(86) PCT No.: PCT/CN2023/080386

§ 371 Date: Sep. 6, 2024

(30) Foreign Application Priority Data
Mar. 10, 2022 (CN) 202210233546.6

(51) Int. Cl. A61K 40/42; A61K 40/11; A61K 40/31; A61P 35/00; C07K 14/725; C07K 16/30; C12N 9/22; C12N 15/11; C12N 15/113

(52) U.S. Cl. CPC A61K 40/4254; A61K 40/11; A61K 40/31; A61P 35/00; C07K 14/7051; C07K 16/30; C12N 9/226; C12N 15/11; C12N 15/1138; C07K 2317/622; C12N 2310/11; C12N 2310/20; C12N 2310/531

(57) ABSTRACT

An immune cell with down-regulated cell adhesion capability and the medical use thereof. The immune cell is a tumor killer cell for adoptive immune cell therapy, and has down-regulated cell adhesion capability. The immune cell has better safety for hematological tumors and solid tumors.

### CROSS-REFERENCE TO RELATED APPLICATIONS

This application is the U.S. national phase of International Application No. PCT/CN2023/080386 filed Mar. 9, 2023, which designated the U.S. and claims priority to CN 202210233546.6 filed Mar. 10, 2022, the entire contents of each of which are hereby incorporated by reference.

### REFERENCE TO SEQUENCE LISTING SUBMITTED ELECTRONICALLY

The content of the electronically submitted sequence listing (Name: 6492_0095_Sequence_Listing.xml, Size: 87,328 bytes, and Date of Creation: May 18, 2025) is herein incorporated by reference in its entirety.

### TECHNICAL FIELD

The present disclosure relates to the field of biopharmaceutical technology, and in particular, to an immune cell with down-regulated cell adhesion capability and pharmaceutical use thereof.

### BACKGROUND

In recent years, the development of immunotherapy has brought profound changes to the field of cancer therapies, especially the immune checkpoint therapies represented by PD1/PDL1 pathway inhibitors and adoptive cell therapies represented by CAR-T. The adoptive cell therapies include TIL, NK, TCR-T, CAR-T, etc. Among them, the CAR-T therapy targeting CD19 has exhibited excellent clinical efficacy in B-cell tumors. So far, five CAR-T products have been approved in the US by the FDA for the treatment of B-cell leukemia or lymphoma, and two have now been available for patients in China.

The concept of chimeric antigen receptors (CARs) was first proposed by Zelig Eshhar, et al. The CAR-T can highly specifically target tumor antigens without relying on the major histocompatibility complex (MHC). The structure of CAR is composed of an extracellular single-chain variable fragment (scFv), a transmembrane region, a co-stimulatory region, and an intracellular signaling region. The first-generation CARs contain only one signaling unit, mainly derived from the CD3ζ or FcRγ subunit. The second-generation CARs, where a co-stimulatory factor is introduced on the basis of the first-generation CARs, comprise a co-stimulatory signaling domain and a signaling domain. In the third-generation, CAR-T cells, two co-stimulatory factors such as CD28 and 4-1BB are introduced, which enhances the anti-tumor effects of the T cells. On the basis of the existing antibody recognition region and signaling region, in the fourth generation CARs (TRUCK T cells), one or more constitutive or inducible expression components are added to enable the CAR-T cells to express specific proteins, thus enhancing the viability of CAR-T cells, promoting the infiltration of T cells into tumor tissues, and resisting the tumor inhibitory microenvironment.

CAR-T therapies, represented by CD19 CAR-T targeting CD19 antigen, have exhibited excellent efficacy in hematological tumors. CD19 CAR-T has been approved in the treatment of adult B-cell type acute lymphoma (ALL), chronic lymphoma (CLL), non-Hodgkin's lymphoma (NHL), and other diseases, and most of the products have a complete response rate of 70% or greater in clinical applications.

Although CAR-T has made major breakthroughs in hematological tumors, the progress in treating solid tumors is limited. One of the important reasons is that the targets in solid tumors, tumor-associated antigens (TAAs), are expressed to some extent in normal tissues despite the high expression in tumor tissues. The expression of TAAs in normal tissues greatly limits the application of CAR-T in solid tumors. After encountering the target antigen in the human body, CAR-T cells will expand and kill both tumor cells and normal cells with target antigens. Therefore, the expression of target antigens in normal tissues leads to on-target off-tumor toxicity during CAR-T treatments, resulting in normal tissue damage and even serious adverse reactions.

### SUMMARY OF THE INVENTION

In a first aspect, the present disclosure relates to an immune cell for use as a tumor killer cell in an adoptive immune cell therapy with down-regulated cell adhesion capability.

In a second aspect, the present disclosure relates to a nucleic acid construct, comprising a first expression cassette and a second expression cassette, wherein
the first expression cassette is used to express an antagonistic component of a cell adhesion molecule;
the second expression cassette is used to express a chimeric antigen receptor;
the first expression cassette and the second expression cassette are located on the same nucleic acid construct, or on different nucleic acid constructs; and
the antagonistic component is a molecule that can specifically inhibit the transcription or translation of the cell adhesion molecule, or can specifically inhibit the expression or activity of the cell adhesion molecule protein.

In a third aspect, the present disclosure relates to a method for preparing the immune cell as described above, comprising, transferring the nucleic acid construct as described above into an immune cell for expression; or treating an immune cell with an antagonistic component, and transferring the second expression cassette into the immune cell before, after, or during the treatment.

In a fourth aspect, the present disclosure relates to a pharmaceutical composition, comprising the immune cell as described above.

In a fifth aspect, the present disclosure relates to a pharmaceutical combination product or pharmaceutical composition, comprising: a tumor killer cell for use in an adoptive immune cell therapy and an antagonistic component, wherein
the antagonistic component can induce reduced expression or inhibited functionality of a cell adhesion molecule on the surface of a tumor killer cell, so as to give the immune cell as described above.

In a sixth aspect, the present disclosure relates to use of the immune cell as described above in preparing a medicament for preventing and/or treating tumor.

The immune cell provided by the present disclosure can effectively control the tumor with corresponding target antigens in animal models, and can significantly reduce the toxicity of immune cells to normal tissues and effectively resist tumor metastasis.

In a seventh aspect, the present disclosure relates to use of a tumor killer cell for an adoptive immune cell therapy in combination with an antagonistic component in preparing a medicament for preventing and/or treating a tumor, wherein
the tumor killer cell for the adoptive immune cell therapy and the antagonistic component are as defined above.

In an eighth aspect, the present disclosure relates to a method for preventing and/or treating a tumor, comprising: administering to a subject an effective amount of the pharmaceutical composition according to the fourth aspect or the pharmaceutical combination product or pharmaceutical composition according to the fifth aspect.

The immune cell possesses a longer circulation time in the blood, reduced adhesion and vascular penetration ability, and lower toxicity, thereby exhibiting better efficacy and better safety in both hematological tumors and solid tumors and thus good application prospects.

### BRIEF DESCRIPTION OF THE DRAWINGS

In order to more clearly illustrate the specific embodiments of the present disclosure or embodiments in the prior art, the drawings used for the specific embodiments and the prior art are briefly introduced below. It is obvious that the drawings in the following description are only illustrative of some embodiments of the present disclosure, while other drawings can be obtained by those of ordinary skill in the art without creative efforts.

FIG. 1 shows a structural schematic of the plasmid according to one example of the present disclosure; the upper and lower strands of the double stranded DNA shown consist of the nucleotide sequence of SEQ ID NO: 87 and 88, respectively;

FIG. 2 shows a structural schematic of the shRNA expression cassette according to one example of the present disclosure;

FIG. 3 shows the transduction rates (CAR+) of the prepared untransduced T (UNT) cells and CAR-T-Pericruiser® cells as determined by flow cytometry according to one example of the present disclosure;

FIG. 4 shows the adhesion-associated molecules knocking down efficiency by means of shRNA or Crispr method as determined by flow cytometry according to one example of the present disclosure;

FIG. 5 shows the adhesion assay results of CAR-T-Pericruiser® cells (shRNA method) according to one example of the present disclosure;

FIG. 6 shows the in vitro killing assay results of EpCAM-CAR-T-Pericruiser® (shRNA method) and UNT cells against target cells HCT116, MKN45 (with high EpCAM expression), and MIA-Paca2 (with low EpCAM expression) according to one example of the present disclosure;

FIG. 7 shows the acute toxicity study of EpCAM-CAR-T cells and EpCAM-CAR-T-Pericruiser® cells (shRNA method) in treating M-NSG mouse 4T1 tumor model according to one example of the present disclosure;

FIG. 8 shows the effective control of metastases by EpCAM-CAR-T-Pericruiser® cells (shRNA method) according to one example of the present disclosure, wherein A: tumor signal images in mice after EpCAM-CAR-T-Pericruiser® or UNT cell treatment at various time points; B: images of tumor metastases in the liver, kidneys, and lungs of mice receiving EpCAM-CAR-T-Pericruiser® or UNT cells at the end of study; C: tumor fluorescence intensity at various time points;

FIG. 9 shows the good safety of human EpCAM-CAR-T-Pericruiser® cells (shRNA method) in the M-NSG mouse HCT116 xenograft tumor model with no significant difference in body weight and survival after 40 days compared with untransduced T (UNT) cells according to one example of the present disclosure;

FIG. 10 shows the effective control of tumors in situ and tail vein metastases by EpCAM-CAR-T-Pericruiser® cells (shRNA method) according to one example of the present disclosure, wherein A: tumor signals at various time points after the cell treatments; B: signal diagram of lung and liver metastases in the CAR-T and UNT treatment groups at the end of study; C: lung and liver metastases in the CAR-T and UNT treatment groups at the end of study;

FIG. 11 shows the effective control of abdominal metastases by EpCAM-CAR-T-Pericruiser® cells (shRNA method) in combination with chemotherapy according to one example of the present disclosure;

FIG. 12 shows the effective control of xenograft NALM-6-Luc hematological tumor by CD19-CAR-T-Pericruiser® cells (shRNA method) according to one example of the present disclosure, wherein A: the CD19-CAR-T-Pericruiser® cell treatment group had a longer survival; B: the CD19-CAR-T-Pericruiser® cell treatment group showed weaker tumor fluorescence; C: no significant weight loss was observed in the CD19-CAR-T-Pericruiser® cell treatment group;

FIG. 13 shows the in vitro and in vivo studies of EpCAM-CAR-T cells in combination with natalizumab and efalizumab according to one example of the present disclosure, wherein A: the combination with natalizumab and efalizumab did not affect the cell killing functionality of CAR-T cells in vitro; B: in the CAR-T/antibody combination treatment groups, the binding of CAR-T cells to ICAM-1 and VCAM-1 was blocked, and the adhesion capability of CAR-T cells to vascular cells HUVEC was reduced; C: in the acute toxicity study, the weight loss in mice was reduced in the CAR-T/antibody combination treatment groups; D: in the acute toxicity study, the CAR-T/antibody combination treatment groups exhibited significantly prolonged survivals; E: in the acute toxicity study, no T cell infiltration was observed in the lungs, liver, kidneys, and pancreas of mice in the CAR-T/antibody combination treatment groups;

FIG. 14 shows the in vitro verification of targeted knockout of PSGL1, CD11a, CD18, CD29, or CD49d by sgRNA in EpCAM-CAR-T cells according to one example of the present disclosure, wherein A: the sgRNA targeting PSGL1, CD11a, CD18, CD29, or CD49d effectively knocked out the target genes in vitro; B: knockout of PSGL1, CD11a, CD18, CD29 and CD49d in EpCAM-CAR-T cells did not affect the killing ability of CAR-T; C: knockout of CD11a and CD18 inhibited the adhesion of CAR-T cells to ICAM-1, knockout of CD29 and CD49d inhibited the adhesion of CAR-T cells to VCAM-1, and knockout of PSGL1 inhibited the adhesion of CAR-T cells to P-selectin;

FIG. 15 shows the in vitro and in vivo functional verification of cells with dual knockout of PSGL1, CD11a, CD18, CD29, and CD49d according to one example of the present disclosure, wherein A: the knockout combinations had no effect on CAR structure or expression; B: the dual knockout combinations effectively knocked out the two corresponding genes; C: the dual knockout combinations did not affect the killing effects of CAR-T cells on the target cell HCT116; D: the knockout combinations inhibited the adhesion of CAR-T cells to P-selectin, ICAM-1, or VCAM-1; E: the knockout combinations inhibited the adhesion of CAR-T cells to vascular endothelial cell HUVEC;

FIG. 16 shows the in vitro and in vivo functional verification of cells with individual or dual adhesion gene knockout according to one example of the present disclosure; A: verification of the knockout efficiency of individual knockout of CD29 or CD49d and dual knockout of CD29 and CD49d; B: the animal acute toxicity study showed that the individual knockout of CD29 or CD49d and dual knockout of CD29 and CD49d partially inhibited CAR-T toxicity; C: the knockout of CD11a-CD49d or CD18-CD49d did not affect CAR expression; D: verification of the knockout efficiency of CD11a-CD49d and CD18-CD49d in CAR-T cells; E: the knockout of CD11a-CD49d or CD18-CD49d in CAR T cells effectively inhibited the on-target off-tumor toxicity of CAR-T cells and reduced the weight loss in mice;

FIG. 17 shows the in vitro functional assay of cells with PSGL1, CD11a, CD18, CD29, or CD49d knockdown according to one example of the present disclosure, wherein A: knockdown efficiency detection of PSGL1, CD11a, CD18, CD29, and CD49d; B: CAR expression on CAR-T cell surface with PSGL1, CD11a, CD18, CD29, or CD49d knockdown; C: apoptosis of cells with PSGL1, CD11a, CD18, CD29, or CD49d knockdown; D: memory phenotype of CAR-T cells with PSGL1, CD11a, CD18, CD29, or CD49d knockdown; E: exhuasion phenotype of CAR-T cells with PSGL1, CD11a, CD18, CD29, or CD49d knockdown; F: target cell killing assay of CAR-T cells with PSGL1, CD11a, CD18, CD29, or CD49d knockdown;

FIG. 18 shows the in vivo and in vitro function assay of EpCAM CAR-T cells with multi-knockdown of PSGL1, CD11a, CD18, CD29, and CD49d according to one example of the present disclosure, wherein A, B, C, and D: knockdown efficiency and CAR expression assays of cells with multiple shRNA targeting PSGL1, CD11a, CD18, CD29, and CD49d; E: multi-knockdown of PSGL1, CD11a, CD18, CD29, and CD49d did not affect the killing effects of CAR-T cells on target cell HCT11M; F: multi-knockdown of PSGL1, CD11a, CD18, CD29, and CD49d inhibited the adhesion of CAR-T-mEpCA cells to HUVECs;

FIG. 19 shows the reduced in vivo toxicity of EpCAM CAR-T cells with multi-knockdown of PSGL1, CD11a, CD18, CD29, and CD49d in mice according to one example of the present disclosure, wherein A: multi-knockdown of PSGL1, CD11a, CD18, CD29, CD49d in CAR-T cells inhibited the on-target off-tumor toxicity of the CAR-T cells; B: multi-knockdown of PSGL1, CD11a, CD18, CD29, and CD49d prolonged the survival in mice;

FIG. 20 shows the in vivo CAR-T cell functional assay of human EpCAM CAR-T cells with multi-knockdown of PSGL1, CD11a, CD18, CD29, and CD49d according to one example of the present disclosure, wherein A and B: human EpCAM CAR-T cells with multi-knockdown of PSGL1, CD11a, CD18, CD29, and CD49d effectively inhibited the venous metastasis of tumors; C and D: human EpCAM CAR-T cells with multi-knockdown of PSGL1, CD11a, CD18, CD29, and CD49d effectively eliminated solid tumors.

### DETAILED DESCRIPTION

References to embodiments of the present disclosure are provided in detail by means of one or more examples below. The examples are provided for illustrating rather than limiting the present disclosure. Actually, it is obvious to those skilled in the art that many modifications and variations can be made to the present disclosure without departing from the scope or spirit of the present disclosure. For example, a feature stated or described as part of one embodiment may be used in another embodiment to produce a further embodiment.

Unless otherwise stated, all terms (including technical and scientific terms) used to disclose the present disclosure have the same meaning as would normally be understood by those of ordinary skill in the art. With further guidance, subsequent definitions are used to better understand the teachings of the present disclosure. The terms used in the specification of the present disclosure are for the purpose of describing the specific examples only and are not intended to limit the present disclosure.

The term “and/or” or “or/and” as used herein includes any one of two or more relevant listed items, and any and all combinations of relevant listed items. The any and all combinations include a combination of any two or more or all of the relevant listed items. It will be appreciated that in the present application, when at least three items are connected by at least two combinations of conjunctions selected from “and/or” and “or/and”, the embodiment undoubtedly encompasses the instance where both are connected by logical “and” and the instance where both are connected by logical “or”. For example, “A and/or B” includes three parallel instances, i.e., A, B, and A+B. For another example, an embodiment of “A, and/or, B, and/or, C, and/or, D” includes any one item of A, B, C, and D (the instance where the items are connected by logic “or”), and also includes any and all of the combinations of A, B, C, and D. That is, combinations of any two or three of A, B, C, and D, as well as the combination of A, B, C, and D (the instance where the items are connected by logic “and”) are included.

The terms “comprise”, “contain”, and “include” as used herein are synonyms, and are inclusive or open-ended but not exclusive of additional or uncited members, elements, or procedures.

A numeral range represented by endpoints as used herein includes all values and fractions within the range, as well as the endpoint.

A concentration value, as used herein, includes fluctuations within a certain range. For example, it may fluctuate within a corresponding precision range. For example, for 2%, a fluctuation within the range of ±0.1% may be permitted. For greater values or those that require no fine controls, larger fluctuations may also be permitted. For example, for 100 mM, fluctuations within the range of ±1%, ±2%, ±5%, etc, may be permitted. In terms of molecular weight, a fluctuation within the range of ±10% is permitted.

In the present disclosure, the description related to “multiple”, unless otherwise defined, means 2 or more.

In the present disclosure, the technical features described in an open-ended manner include a closed-ended embodiment consisting of the enumerated features and an open-ended embodiment comprising the enumerated features.

In the present disclosure, the term “preferred”, “preferable”, or “preferably” only describes embodiments or examples with better results and should not be construed as limitations to the protection scope of the present disclosure. In the present disclosure, the term “optional” or “optionally” refers to non-essential, i.e., being selected from either of the two parallel schemes of “presence” or “absence”. If there is more than one “optional” in an embodiment, the “optional” are independent unless otherwise specified or contradicted or constrained by each other.

In a first aspect, the present disclosure relates to an immune cell for use as a tumor killer cell in an adoptive immune cell therapy with down-regulated cell adhesion capability.

The “down-regulated cell adhesion capability” is relative to wild-type immune cells. The adhesion capability includes the adhesion between cells and the adhesion of cells to their surroundings (such as the cytoplasmic matrix). The “down-regulated cell adhesion capability” may be further defined as any preventive and/or interventional measure, method, and/or process that prevents, minimizes, reduces, affects, mitigates, or alters the rolling, binding, adhesion, migration, or interaction of an immune cell with other cells, such as vascular endothelial cells.

In some embodiments, the immune cell comprises a cell adhesion molecule with reduced expression or inhibited functionality.

In the present disclosure, the cell adhesion molecule (CAM) is a protein that is located on the surface of an immune cell and involved in a binding process called cell adhesion to other cells or the extracellular matrix (ECM). Such proteins, usually transmembrane proteins, consist of three domains: one for intracellular interactions with the cytoskeleton, one transmembrane (on cell surface), and one for extracellular interactions or interactions with either other cell adhesion molecules of the same type (homophilic binding) or other cell adhesion molecules or the extracellular matrix (heterophilic binding). The cell adhesion molecule may include one or more of IgSF CAM, integrin, cadherin, selectin, and lymphocyte homing receptor.

The IgSF CAM includes, for example, one or more of N-CAM (myelin protein zero), intercellular adhesion molecules (e.g., ICAM-1 and ICAM5), VCAM-1, PE-CAM, L1 protein family (e.g., L1-CAM, NRCAM, NFASC, and CHL1), nectin (e.g., PVRL1, PVRL2, and PVRL3), etc.

The integrin includes, for example, one or more of LFA-1 (CD11a+CD18), integrin alphaXbeta2 (CD11c+CD18), macrophage-1 antigen (CD11b+CD18), VLA-4 (CD49d+CD29), glycoprotein IIb/IIIa (ITGA2B+ITGB3), etc.

The cadherin includes, for example, one or more of classical cadherins (e.g., CDH1 (gene), CDH2, and CDH3 (gene)), desmosomes (desmogleins (desmoglein-1, desmoglein-2, desmoglein-3, and desmoglein-4) and desmocollins (DSC1, DSC2, and DSC3)), procadherins (e.g., PCDH1 and PCDH15), T-cadherin, CDH4, VE-cadherin, CDH6, CDH8, CDH11, CDH12, CDH15, CDH16, CDH17, CDH9, and CDH10.

The selectin includes, for example, one or more of E-selectin, L-selectin, and P-selectin.

The lymphocyte homing receptor includes, for example, CD44 and L-selectin.

The cell adhesion molecule may also include one or more of carcinoembryonic antigens, CD22, CD24, CD44, CD146, and CD164.

In some preferred embodiments, the cell adhesion molecule includes at least one, e.g., one, two, or more, of PSGL1, CD44, CD11a, CD18, CD49d, CD29, and CXCR4.

For example, the cell adhesion molecule includes:
PSGL1 and CD18; or
PSGL1 and CD49d; or
CD11a and CD18; or
CD11a and CD29; or
CD11a and CD49d; or
CD18 and CD49d; or
CD29 and CD49d; or
PSGL1, CD11, and CD49d; or
PSGL1, CD18, and CD49d.

In some embodiments, at least part of the cell adhesion molecule of the immune cell is knocked down or knocked out.

In some embodiments, at least part of the cell adhesion molecule of the immune cell is blocked by a neutralizing antibody.

In some embodiments, the adoptive immune cell therapy is an NK therapy, a LAK therapy, a DC therapy, a CIK therapy, a TIL therapy, a DC-CIK therapy, a CAR-T therapy, a TCR-T therapy, a CAR-NK therapy, or a TCR-NK therapy.

In some embodiments, the immune cell is not treated or stimulated by a CD3 antibody and/or a CD19 antibody.

As used herein, the “chimeric antigen receptor (CAR)” refers to a fusion protein containing an extracellular domain capable of binding to an antigen, a transmembrane domain derived from a polypeptide different from the polypeptide where the extracellular domain is derived, and at least one intracellular domain. The “chimeric antigen receptor (CAR)” is sometimes referred to as “chimeric receptor”, “T-body”, or “chimeric immune receptor (CIR)”. The term “extracellular domain capable of binding to an antigen” refers to any oligopeptide or polypeptide capable of binding to a specific antigen. The “intracellular domain” refers to any oligopeptide or polypeptide known to function in cells as a domain that transmits signals to activate or inhibit a biological process.

As used herein, the “region” or “domain” in the chimeric antigen receptor refers to a region of a polypeptide that can be folded into a specific structure independently of other regions. Such “regions” or “domains” may be sequences of mouse or other animal origins, preferably a human sequence.

As used herein, the “antigen-recognition domain” refers to a domain that can specifically recognize and bind to an antigen, including but not limited to: single or tandem structures consisting of a single-chain variable fragment, an alpaca antibody, a ligand, etc., each recognizing one or two antigenic targets. The “single-chain variable fragment (scFv)” refers to a single-chain polypeptide that is derived from an antibody and retains the ability to bind to an antigen. Examples of scFv include antibody polypeptides formed by recombinant DNA technology in which the Fv regions of the heavy (H) chain and light (L) chain fragments of immunoglobulins are linked by a spacer sequence. A variety of methods for preparing scFv are known, including those described in the following documents: U.S. Pat. No. 4,694,778; Science, Vol. 242, pp. 423-442 (1988); Nature, Vol. 334, p. 54454 (1989); Science, Vol. 242, pp. 1038-1041 (1988).

In some embodiments, the immune cell expresses a chimeric antigen receptor, and the chimeric antigen receptor comprises an extracellular antigen-recognition domain for recognizing a tumor antigen.

In some embodiments, the antigen-recognition domain specifically recognizes a tumor antigen. The “tumor antigen” is a biomolecule with antigenicity, whose expression is recently recognized to be associated with cell carcinogenesis. Assays, for example, immunological assays of tumor antigens can be used to distinguish cancerous cells from their parent cells. The tumor antigen can be an antigen of a solid tumor or a hematological tumor. The tumor antigen in the present disclosure includes tumor-specific antigens (antigens present only in tumor cells but not in other normal cells) and tumor-associated antigens (antigens also present in other organs and tissues or in heterologous and allogeneic normal cells, or antigens expressed during the development and differentiation processes). The antigen-recognition domain preferably specifically recognizes and binds to at least one of the following antigen molecules:
alpha-fetoprotein, alpha-actinin-4, A3, an antigen specific to an A33 antibody, ART-4, B7, B7H3, Ba 733, BAFF-R, BAGE, BCMA, BrE3 antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD56, CD59, CD64, CD66a/b/c/e, CD67, CD70, CD70L, CD74, CD79a, CD79b, CD80, CD83, CD95, CD117, CD123, CD126, CD132, CD133, CD138, CD147, CD154, CD319, CD371, CDC27, CDK-4/m, CDKN2A, CLL1, CTLA4, CXCR4, CXCR7, CXCL12, HIF-1α, colon-specific antigen-p (CSAp), CEA, CEACAM-6, c-Met, DAM, EGFR, EGFRvIII, EGP-1, EGP-2, ELF2-M, EpCAM, fibroblast growth factor, Flt-1, Flt-3, folate receptor, G250 antigen, GAGE, gp100, GRO-β, GPRC5D, HLA-DR, HM1.24, human chorionic gonadotropin (HCG) and a subunit thereof, HER2/neu, HMGB-1, hypoxia-inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor 1, Igκ, IL1RAP, Lewis Y, LMP1, KC4 antigen, KS-1 antigen, KS1-4, Le-Y, LDR/FUT, macrophage migration inhibitory factor, MAGE, MAGE-3, MART1, MART-2, NY-ESO-1, TRAG-3, mCRP, MCP-1, MMG49, MIP-1A, MIP-1B, MIF, MUC1, MUC2, MUC3, MUC4, MUC5ac, MUC13, MUC16, MUM-1/2, MUM-3, NCA66, NCA95, NCA90, NKG2D ligand, pancreatic mucin, PD-L1, placental growth factor, p53, PLAGL2, prostatic acid phosphatase, PSA, PRAME, PSMA, PlGF, ILGF, ILGF-1R, IL-6, IL-25, RS5, RANTES, ROR1, T101, SAGE, S100, survivin, TAC, TAG-72, tenascin, TRAIL receptor, TNF-alpha, Tn antigen, Thomsen-Friedenreich antigen, tumor necrosis antigen, VEGFR, ED-B fibronectin, WT-1, 17-1A antigen, complement components C3, C3a, C3b, C5a and C5, angiogenesis markers, bc1-2, bc1-6, Kras, oncogenic markers and oncogenic gene products.

In some embodiments, the antigen-recognition domain is a single-chain antibody (preferably scFv).

The single-chain antibody may be a chimeric, humanized, or human antibody fragment that recognizes an antigen of a tumor.

In some embodiments, the chimeric antigen receptor further comprises a hinge region, a transmembrane domain, and an intracellular signaling region.

In some embodiments, the hinge region is selected from a hinge region of CD8α or CD28.

In some embodiments, the transmembrane domain is selected from one of the α, β, or ζ chain of a T-cell receptor, CD28, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244 and 2B4), CD84, CD96 (Tactile), CEACAM1, CRTAM, Ly9 (CD229), CD160 (BY55), PSGL1, CD100 (SEMA4D), SLAMF6 (NTB-A and Ly 108), SLAM (SLAMF1, CD150, and IPO-3), BLAME (SLAMF8), SELPLG (CD162), LTBR, PAG/Cbp, NKp44, NKp30, NKp46, NKG2D, and NKG2C, preferably, the transmembrane region of any one of the above.

In some embodiments, the intracellular signal transduction region is selected from any one of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand specifically binding to CD83, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244 and 2B4), CD84, CD96 (Tactile), CEACAM1, CRTAM, Ly9 (CD229), CD160 (BY55), PSGL1, CD100 (SEMA4D), CD69, SLAMF6 (NTB-A and Ly108), SLAM (SLAMF1, CD150, and IPO-3), BLAME (SLAMF8), SELPLG (CD162), LTBR, LAT, GADS, SLP-76, PAG/Cbp, NKp44, NKp30, NKp46, PKCθ, FccRIγ, ZAP70, and CD3ζ, or any combination thereof, preferably, the co-stimulatory region of CD28 and CD3ζ.

In some embodiments, the immune cell is a T cell, a B cell, an NK cell, or a DC cell.

In some embodiments, the T cell is any one of a helper T cell, a cytotoxic T cell, a memory T cell, a regulatory T cell, a MAIT cell, or a γδT cell.

In a second aspect, the present disclosure further relates to a nucleic acid construct, comprising a first expression cassette and a second expression cassette, wherein
the first expression cassette is used to express an antagonistic component of a cell adhesion molecule;
the second expression cassette is used to express a chimeric antigen receptor; and
the antagonistic component is a molecule that can specifically inhibit the transcription or translation of the cell adhesion molecule, or can specifically inhibit the expression or activity of the cell adhesion molecule protein;

The cell adhesion molecule and the chimeric antigen receptor are as defined above.

In the present disclosure, the nucleic acid construct refers to a sequence that contains a replication system as well as a sequence capable of transcribing and translating a polypeptide-coding sequence within a given target cell.

When the nucleic acid construct enables the expression of the protein encoded by the inserted polynucleotide, it is also referred to as a vector. The vector can be introduced into a host cell by transformation, transduction, or transfection, such that the genetic substance elements carried by the vector can be expressed in the host cell. Vectors are well known to those skilled in the art, including but not limited to: plasmids; phagemids; cosmids; artificial chromosomes such as yeast artificial chromosome (YAC), bacterial artificial chromosome (BAC), or P1-derived artificial chromosome (PAC); phages such as lambda phages or M13 phages; and animal viruses. Animal viruses that can be used as vectors include, but are not limited to, retroviruses (including lentiviruses), adenoviruses, adeno-associated viruses, herpes viruses (e.g., herpes simplex virus), poxviruses, baculoviruses, papillomaviruses, and papovaviruses (e.g., SV40). In some embodiments, the vector of the present disclosure comprises regulatory elements commonly used in genetic engineering, such as enhancers, promoters, internal ribosome entry sites (IRESs), and other expression control elements (e.g., transcription termination signals, or polyadenylation signals and poly U sequences, etc.). Furthermore, the vector further comprises a nucleotide sequence encoding a detectable marker in a cell; the detectable marker is, for example, green fluorescent protein (GFP).

In the present disclosure, the first expression cassette and the second expression cassette may be located on the same nucleic acid construct or on different nucleic acid constructs (that is, more than one nucleic acid construct may be present).

In some embodiments, the first expression cassette is connected to the second expression cassette.

The expression cassettes may have conventional elements that facilitate the expression of a target gene therein, e.g., promoters, terminators, or enhancers.

It is easily understood that, according to the content recorded in the present disclosure, the inhibition of the expression of the cell adhesion molecule gene at the protein level or mRNA level will be effective. The inhibition may be either partially reduced expression or silenced expression of the cell adhesion molecule gene.

The antagonistic component shall be interpreted as including, but not limited to, a nucleic acid molecule, an antibody drug, and an interfering lentivirus.

In some embodiments, the antagonistic component may also be a nuclease used to knock down or knock out the cell adhesion molecule, such as restriction endonuclease, homing endonuclease, meganuclease, TAL effector nuclease (TALEN), zinc finger nuclease, and CRISPR-related nuclease.

In some embodiments, the nucleic acid molecule is a small nucleic acid molecule; for example, the nucleic acid molecule is selected from: an antisense oligonucleotide, dsRNA, microRNA, siRNA, shRNA, and a nucleic acid encoding the CRISPR system (including sgRNA and a nucleic acid encoding the Cas enzyme), preferably shRNA.

In the present disclosure, the shRNA, or small hairpin RNA or short hairpin RNA, is an RNA sequence with a tight hairpin turn. It consists of a sense strand fragment, an antisense strand fragment, and a stem-loop structure connecting the sense strand fragment and the antisense strand fragment, and is often used for RNA interference to silence the expression of target genes. The sequences of the sense strand and the antisense strand are complementary, and the sense strand fragment has a sequence of 10 to 30 continuous nucleotides identical to that of the cell adhesion molecule gene; preferably, the sense strand fragment has a sequence of 15 to 27 continuous nucleotides identical to that of the cell adhesion molecule gene; more preferably, the sense strand fragment has a sequence of 19 to 23 continuous nucleotides identical to that of the cell adhesion molecule gene, or optionally, a sequence of 19, 20, or 21 consecutive nucleotides identical, or a sequence hybridized with the above sequences with very high stringency. The hairpin structure of shRNA can be cleaved into siRNAs by cellular mechanisms, and the siRNAs then bind to an RNA-induced silencing complex (RISC) capable of binding to the target mRNA and degrading same.

The sequence of the stem-loop structure of the shRNA may be a general choice in the art, for example, a sequence selected from any one of the following: UUCAAGAGA, AUG, CCC, UUCG, CCACC, CUCGAG, AAGCUU, and CCACACC.

In a third aspect, the present disclosure further relates to a method for preparing the immune cell as described above, comprising, transferring the nucleic acid construct as described above into an immune cell for expression; or
treating an immune cell with the antagonistic component described above, and transferring the second expression cassette into the immune cell before, after, or during the treatment.

In some preferred embodiments, a nucleic acid construct comprising the second expression cassette is first transferred into the immune cell, and after an interval of 1 to 4 days (such as 2 or 3 days), the second expression cassette is transferred.

In a fourth aspect, the present disclosure further relates to a pharmaceutical composition, comprising the immune cell as described above.

In a fifth aspect, the present disclosure further relates to a pharmaceutical combination product or pharmaceutical composition, comprising: a tumor killer cell for use in an adoptive immune cell therapy and an antagonistic component, wherein
the antagonistic component can induce reduced expression or inhibited functionality of a cell adhesion molecule on the surface of a tumor killer cell, so as to give the immune cell as described above.

The “pharmaceutical combination product” means that at least two ingredients are packaged in separate containers, but not as a mixture.

The pharmaceutical composition/pharmaceutical combination product may also comprise a pharmaceutically acceptable carrier. As used herein, the “pharmaceutically acceptable carrier” includes any material that, when combined with an active ingredient, allows the ingredient to remain biologically active and not react with the subject's immune system. Examples include, but are not limited to, any of standard pharmaceutical carriers (e.g., phosphate-buffered saline, lactose, glucose, sucrose, sorbitol, mannitol, starch, microcrystalline cellulose, polyvinylpyrrolidone, cellulose, water, and emulsions (such as oil/water emulsions)) and various wetting agents. Exemplary diluents for aerosol or parenteral administration are phosphate-buffered saline (PBS) or normal saline (0.9%). Compositions containing such carriers are prepared by well-known conventional methods (see, e.g., Remington's Pharmaceutical Sciences, 18^{th} edition, A. Gennaro, eds., Mack Publishing Co., Easton, PA, 1990; and R Remington, The Science and Practice of Pharmacy, 21^{st} edition, Mack Publishing, 2005).

When the pharmaceutical composition/pharmaceutical combination product is used to prevent or treat a tumor in a subject, an effective dose of the pharmaceutical composition/pharmaceutical combination product shall be administered to the subject. By this method, the growth, proliferation, recurrence, and/or metastasis of the tumor is inhibited. Furthermore, at least 10%, 20%, 30%, 40%, 50%, 60%, 70%, 80%, 90%, 95%, or 99% of the growth, proliferation, recurrence, and/or metastasis of the tumor is inhibited.

The subject of the present disclosure may be an animal, preferably a mammal including, but not limited to, primates (humans and monkeys), pigs, horses, cows, donkeys, camels, dogs, cats, rabbits, rodents (including mice, rats, and guinea pigs), etc.

In a sixth aspect, the present disclosure further relates to use of the immune cell as described above in preparing a medicament for preventing and/or treating a tumor.

In a seventh aspect, the present disclosure further relates to use of a tumor killer cell for an adoptive immune cell therapy in combination with an antagonistic component in preparing a medicament for preventing and/or treating a tumor, wherein
the tumor killer cell for the adoptive immune cell therapy and the antagonistic component are as defined in the fifth aspect above.

In the present disclosure, the “tumor” includes a solid tumor or a hematological tumor, including, for example, a tumor derived from a lesion at any of the following: bones, blood, bone junctions, muscles, lungs, tracheas, heart, spleen, arteries, veins, capillaries, lymph nodes, lymph vessels, lymph fluid, mouth, pharynx, esophagus, stomach, duodenum, small intestine, colon, rectum, anus, appendix, liver, gallbladder, pancreas, parotid glands, sublingual glands, kidneys, ureters, bladder, urethra, ovaries, fallopian tubes, uterus, vagina, vulva, scrotum, testes, vasa deferentia, penis, eyes, ears, nose, tongue, skin, brain, brainstem, medulla oblongata, spinal cord, cerebrospinal fluid, nerves, thyroid, parathyroid glands, adrenal glands, pituitary gland, pineal gland, islet, thymus, gonads, sublingual glands, and parotid glands. In particular, the preferred tumor can be targeted, for example, cholangiocarcinoma, breast cancer, cervical cancer, chronic lymphocytic leukemia, chronic myelogenous leukemia, colorectal cancer, endometrial cancer, esophageal cancer, gastric cancer, head and neck cancer, Hodgkin's lymphoma, lung cancer, medullary thyroid cancer, non-Hodgkin's lymphoma, multiple myeloma, renal cancer, ovarian cancer, pancreatic cancer, neuroglioma, melanoma, liver cancer, prostate cancer, and urinary bladder cancer.

Preferably, the medicament is used to treat tumor metastasis.

The present disclosure relates to a method for preventing and/or treating a tumor, comprising: administering to a subject an effective amount of the pharmaceutical composition according to the fourth aspect above or the pharmaceutical combination product or pharmaceutical composition according to the fifth aspect.

In some embodiments, the method is used to treat an in situ lesion and/or a metastasis of the tumor.

In some embodiments, the method is used to reduce an in vivo toxicity of the tumor killer cell (including the tumor killer cells described in the fourth aspect and the fifth aspect).

In some embodiments, the tumor killer cell (including the tumor killer cells described in the fourth aspect and the fifth aspect) retains the ability to clear the tumor.

As used herein, the term “effective amount” refers to a dose of a corresponding component of the term capable of achieving the treatment, prevention, alleviation, and/or remission of a disease or condition described in the present disclosure in a subject.

In some embodiments, the immune cell described above, or the nucleic acid construct described above, or the pharmaceutical composition/pharmaceutical combination product described above are delivered to a tissue of interest by, for example, intramuscular injection, intravenous administration, transdermal administration, intranasal administration, oral administration, or mucosal administration.

It will be appreciated that the envisaged treatment may further comprise administering an additional tumor treatment entity, including viral cancer vaccines (e.g., adenovirus vectors encoding cancer-specific antigens), bacterial cancer vaccines (e.g., non-pyrogenic Escherichia coli expressing one or more cancer-specific antigens), yeast cancer vaccines, N-803 (also known as ALT-803, ALTOR), chemotherapeutics (such as docetaxel, toposide, platinum-based agents, irinotecan, TIC10, or docetaxel), antibodies (e.g., those binding to tumor-associated antigens or patient-specific tumor neoantigens), antitumor electric field therapy, stem cell grafts (e.g., allogeneic or autologous), and tumor-targeting cytokines (e.g., NHS-IL12 and IL-12 conjugated with tumor-targeting antibodies or fragments thereof). In some embodiments, the envisaged treatment further comprises administering a radiation therapy to the patient. In some embodiments, the envisaged treatment further comprises performing a surgery on the patient, such as tumorectomy. In some preferred embodiments, the envisaged treatment further comprises administering a combined chemotherapeutic (e.g., platinum-based agents, preferably platinum, cisplatin, carboplatin, nedaplatin, oxaliplatin, satraplatin, and triplatin tetranitrate) to the patient. The combination with chemotherapy is preferred.

The embodiments of the present disclosure will be described in detail below with reference to the examples. It will be appreciated that these examples are for illustrative purposes only and are not intended to limit the scope of the present disclosure. Procedures without specified conditions in the following examples are preferably conducted with reference to the guidelines provided in the present disclosure, in accordance with experimental manuals or conventional conditions in the art, with reference to other experimental procedures known in the art, or in accordance with conditions recommended by the manufacturer.

In the specific examples below, the measurements for raw material components may have slight errors within the range of measurement accuracy unless otherwise specified. For the temperature and time parameters, acceptable errors from instrument accuracy or operating accuracy are permitted.

The CAR-T-shRNA expression vector constructed in the examples simultaneously achieved the CAR expression and the gene silencing of related cell adhesion molecules, including the above T-cell-related molecules PSLG-1, CD44, CD11a, CD18, CD49d, CD29, etc. The obtained CAR T-X-shRNA (CAR-T-Pericruiser®) can achieve the specific targeting against tumor cells, prevent the CAR-T cells from infiltrating normal tissues, and reduce the toxicity of the CAR-T cells, thus being applicable to the treatment of cancers. Also, the design in the embodiments reduces the infiltration of CAR-T cells into tissues, thereby allowing CAR-T cells to circulate in peripheral blood and thus effectively preventing or treating tumor metastasis. Based on the above characteristics, the present disclosure can be applied to the treatment of solid tumors and hematological tumors. The embodiments feature broad application prospects in, for example, improving the anti-solid tumor toxicity of EpCAM-CAR-T, improving the persistence of CD19 CAR-T, and preventing neurotoxicity.

### EXAMPLE 1: CAR-T-X-SHRNA (CAR-T-PERICRUISER®) PLASMID CONSTRUCTION AND VIRUS PACKAGING

### 1.1 Plasmid Construction

### Procedures

The shRNA sequences for PSLG-1, CD44, CD11a, CD18, CD49d, and CD29 were obtained by online tools such as Sigma or Life Technologies' online shRNA design tools, and SiDirect (http://sidirect2.rnai.jp/) and DSIR (http://biodev.extra.cea.fr/DSIR/DSIR.html), and the Crispr sgRNA sequences were designed by IDG (https://eu.idtdna.com/pages/products/crispr-genome-editing) or other tools.

| ID | shRNA Seed seq/sgRNA seq |
|---|---|
| shCD18-1 | CTCATTAAGAATGCTTACAAT (SEQ ID NO: 1) |
| shCD18-2 | ATCCCATTAATTATATTGTTA (SEQ ID NO: 2) |
| shCD18-3 | GTCCCAGTGGAACAATGATAA (SEQ ID NO: 3) |
| shCD18-4 | GGtGAAGACCtACGAGAAACt (SEQ ID NO: 4) |
| shCD18-5 | GGTGTGACACTGGCTACATTG (SEQ ID NO: 5) |
| shCD18-6 | GCATTCTCCTGCTGGTCATCT (SEQ ID NO: 6) |
| shCD18-7 | GGACCGCTACCTCATCTATGT (SEQ ID NO: 7) |
| shCD18-8 | GAAACCCAGGAAGACCACAAT (SEQ ID NO: 8) |
| shCD18-9 | CTGGTGAAGACCTACGAGAAAC (SEQ ID NO: 9) |
| shCD18-10 | CCAGGAGTGCACGAAGTTCAAG (SEQ ID NO: 10) |
| shCD18-11 | AAGGACATCAGTCTGATTAAAG (SEQ ID NO: 11) |
| shCD18-12 | CGGTGCGTGTTTCCTGTGCAAG (SEQ ID NO: 12) |
| shCD18-13 | CCCCATTAATTATATTGTTAAT (SEQ ID NO: 13) |
| shCD18-14 | GTGCCAATTTATTTACATTTAA (SEQ ID NO: 14) |
| shCD18-15 | TGCCAATTTATTTACATTTAAA (SEQ ID NO: 15) |
| shCD18-16 | CACGTGTATAGAAAAAAAATAA (SEQ ID NO: 16) |
| shCD18-17 | CACGACGGTCATGAACCCCAAG (SEQ ID NO: 17) |
| shCD18-18 | CAGGGTCTTCCTGGATCACAAC (SEQ ID NO: 18) |
| shCD18-19 | CTGCGAGTGTGACACCATCAAC (SEQ ID NO: 19) |
| shCD18-20 | TAGAAAAAAAATAAAACTTCAA (SEQ ID NO: 20) |
| shCD18-21 | CCCGATCACCTTCCAGGTGAAG (SEQ ID NO: 21) |
| shCD11a-1 | GAGCGAGTTTGTGAAAATTCT (SEQ ID NO: 22) |
| shCD11a-2 | AACCAGTAACAAGATGAAAGA (SEQ ID NO: 23) |
| shCD11a-3 | GTGAGGCAAACTTGAGAGTGT (SEQ ID NO: 24) |
| shCD11a-4 | GCACGCCAATGTGACCTGTAA (SEQ ID NO: 25) |
| shCD11a-5 | CCAATCTGTTTCCAGATCAAG (SEQ ID NO: 26) |
| shCD11a-6 | ATGCATGTATTTATCCAATAAA (SEQ ID NO: 27) |
| shCD11a-7 | ACACATGTTGCTGTTGACCAAT (SEQ ID NO: 28) |
| shCD11a-8 | CAGCAGCTATCTCTCAGTGAAC (SEQ ID NO: 29) |
| shCD11a-9 | CGGAAGAGAATGTCTGATCTAA (SEQ ID NO: 30) |
| shPSGL1-1 | ACCAAAAGAGGTCTGTTCATA (SEQ ID NO: 31) |
| shPSGL1-2 | GAGACAGGCCACCGAATATGA (SEQ ID NO: 32) |
| shPSGL1-3 | GGAGAtACAGACCACtCAACC (SEQ ID NO: 33) |
| shPSGL1-4 | GGAAGCACAGACCACICAACC (SEQ ID NO: 34) |
| shPSGL1-5 | CTGGAGATACAGACCACTCAAC (SEQ ID NO: 35) |
| shPSGL1-6 | CCGGACCAATATCCCTCTAAAC (SEQ ID NO: 36) |
| shPSGL1-7 | CCGGAAGCACAGACCACTCAAC (SEQ ID NO: 37) |
| shPSGL1-8 | CACGATAGCGGAATCCTTCAAG (SEQ ID NO: 38) |
| shCD49d-1 | GCAAGTACAGAGCTAGGACAT (SEQ ID NO: 39) |
| shCD49d-2 | GCTCCGTGTTATCAAGATTAT (SEQ ID NO: 40) |
| shCD49d-3 | CGGGAGCAGTAATGAATGCAA (SEQ ID NO: 41) |
| shCD49d-4 | AAGAGTGTTTGTGTACATCAAC (SEQ ID NO: 42) |
| shCD49d-5 | TTGGAGTTATATCAACAGTAAA (SEQ ID NO: 43) |
| shCD49d-6 | TGGATGCAATCTGTAAAGAAAA (SEQ ID NO: 44) |
| shCD49d-7 | ACGACTCTACATGTCAAACTAC (SEQ ID NO: 45) |
| shCD49d-8 | TACAATATAACTACAAATAAAT (SEQ ID NO: 46) |
| shCD49d-9 | TGGAGAAGTCTTAGACTTGAAA (SEQ ID NO: 47) |
| shCD29-1 | CCAAATCATGTGGAGAATGTA (SEQ ID NO: 48) |
| shCD29-2 | TTTGTAGGAAGAGGGATAATA (SEQ ID NO: 49) |
| shCD29-3 | GCCCTCCAGATGACATAGAAA (SEQ ID NO: 50) |
| shCD29-4 | GCCTTGCATTACTGCTGATAT (SEQ ID NO: 51) |
| shCD29-5 | TCCCACAACACTGAATGCAAA (SEQ ID NO: 52) |
| shCD29-6 | AGTGTGGTGTCTGTAAGTGTA (SEQ ID NO: 53) |
| shCD29-7 | TGTCTTGACTCTGATGTATTT (SEQ ID NO: 54) |
| shCtrl | GAGCAAGAGAGCCGGATAAAT (SEQ ID NO: 55) |
| shFLuc | GATTGACAAATACGATTTATC (SEQ ID NO: 56) |
| crRNA-CD29-1 | AUUUAGACAUUUUUACAGGA (SEQ ID NO: 57) |
| crRNA-CD29-2 | UCAUCACAUCGUGCAGAAGU (SEQ ID NO: 58) |
| crRNA-CD29-3 | UUUAGAAGCCUUAAAAAAGA (SEQ ID NO: 59) |
| crRNA-CD29-4 | UUAGAAGCCUUAAAAAAGAA (SEQ ID NO: 60) |
| crRNA-CD49-1 | GUCUCGCUUUAGGCUCCUAG (SEQ ID NO: 61) |
| crRNA-CD49-2 | UCUCGCUUUAGGCUCCUAGU (SEQ ID NO: 62) |
| crRNA-CD49-3 | UGGCAGUGGGCGCACCCACU (SEQ ID NO: 63) |
| crRNA-CD49-4 | UUGGCGAGCCAGUUGGCAGU (SEQ ID NO: 64) |
| crRNA- | GCACTACCAGAGCTAACTCA (SEQ ID NO: 65) |
| scramble | |
| crRNA-CD18-1 | CGTTCAACGTGACCTTCCGG (SEQ ID NO: 66) |
| crRNA-CD18-2 | GCCGGGAATGCATCGAGTCG (SEQ ID NO: 67) |
| crRNA-CD18-3 | TCAGATAGTACAGGTCGATG (SEQ ID NO: 68) |
| crRNA-CD18-4 | CCAGAACUUCACAGGGCCGG (SEQ ID NO: 69) |
| crRNA-CD11a- | CACGTCCAGGTTGTAGCTCG (SEQ ID NO: 70) |
| 1 | |
| crRNA-CD11a- | GCCGGCCTCGAGCTACAACC (SEQ ID NO: 71) |
| 2 | |
| crRNA-CD11a- | CACACGTTCGAGACAGCCCA (SEQ ID NO: 72) |
| 3 | |
| crRNA-CD11a- | GTAGCTCGAGGCCGGCGCTG (SEQ ID NO: 73) |
| 4 | |
| crRNA-PSGL1- | ATCTAGGTACTCATATTCGG (SEQ ID NO: 74) |
| 1 | |
| crRNA-PSGL1- | CAGGAGGAGUUGCAGAGGCA (SEQ ID NO: 75) |
| 2 | |
| crRNA-PSGL1- | CACTCAACCAGTGCCCACGG (SEQ ID NO: 76) |
| 3 | |
| crRNA-PSGL1- | GCTTGCCCGGGACCGGAGAC (SEQ ID NO: 77) |
| 4 | |

Note:
shCtrl and shFLuc were both control shRNAs.

The crRNA was the targeting sequence for the corresponding cell adhesion molecules in the Crispr experiment.

In the plasmid construction, gene fragments of shRNA and different modules in CARs were synthesized by CRO and cloned by conventional molecular cloning techniques.

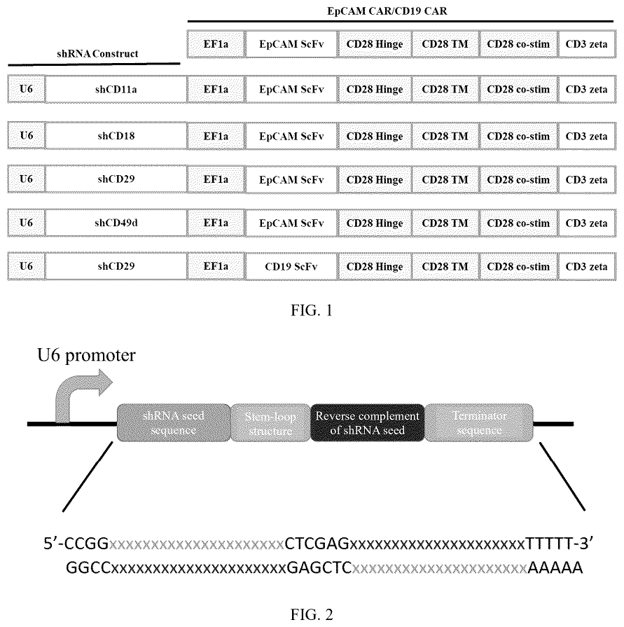

The schematic of the vector construction strategy is shown in FIG. 1, and the CAR structure and component sequences are as follows:

Mouse EpCAM CAR
(SEQ ID NO: 78)
atgcttctcctggtgacaagccttctgctctgtgagttaccacacccagcattcctcctgatcccagaggtgcagctggccgagagcggc
gggggcctggtgcagcccggcagaagcatgaagctgagctgcgccgctagcggcttcaccttcagcaacttccccatggcctgggtgaga
caagcccccaccaagggcctggagtgggtggccaccatcagcacaagcggcggcagcacctactacagagacagcgtgaagggcagattc
accatcagcagagacaacgccaagagcaccctgtacctgcagatgaacagcctgagaagcgaggacaccgccacctactactccacaaga
accctgtacatcctgagagtgttctacttcgactactggggccaaggcgtgatggtgaccgtgagcagcggcgggggcggcagcggcggc
gggggcagcggcggcgggggcagcgacattcagatgacacagagccccgctagcctgagcgctagcctgggcgagaccgtgagcatcgag
tgcctggctagcgagggcatcagcaacgacctggcctggtatcagcagaagagcggcaagagccctcagctgctgatctacgccacaagc
agactgcaagacggcgtgcctagcagattcagcggcagcggcagcggcacaagatacagcctgaagatcagcggcatgcagcccgaggac
gaggccgactacttctgtcagcagagctacaagtacccctggaccttcggcgggggcaccaagctggagctgaagattgaagttatgtat
cctcctccttacctagacaatgagaagagcaatggaaccattatccatgtgaaagggaaacacctttgtccaagtcccctatttcccgga
ccttctaagcccttttgggtgctggtggtggttgggggagtcctggcttgctatagcttgctagtaacagtggcctttattattttctgg
gtgaggagtaagaggagcaggctcctgcacagtgactacatgaacatgactccccgccgccccgggcccacccgcaagcattaccagccc
tatgccccaccacgcgacttcgcagcctatcgctccagagtgaagttcagcaggagcgcagacgcccccgcgtaccagcagggccagaac
cagctctataacgagctcaatctaggacgaagagaggagtacgatgttttggacaagagacgtggccgggaccctgagatggggggaaag
ccgagaaggaagaaccctcaggaaggcctgtacaatgaactgcagaaagataagatggcggaggcctacagtgagattgggatgaaaggc
gagcgccggaggggcaaggggcacgatggcctttaccagggtctcagtacagccaccaaggacacctacgacgcccttcacatgcaggcc
ctgccccctcgctaa
Human EpCAM CAR
(SEQ ID NO: 79)
atgcttctcctggtgacaagccttctgctctgtgagttaccacacccagcattcctcctgatcccacaggttcagctggttcagtctggc
gccgaagtgaagaaacctggcgcctctgtgaaggtgtcctgcaaggcttctggctacacctttaccaactactggatcaactgggtccga
caggctcctggacagggactcgagtggatcggcaacatctaccccagctacatctacaccaattacaatcaagagttcaaggacaaagtg
accctgaccgtggacgagtccacctccaccgcttacatggaactgtccagcctgagatccgaggacaccgccgtgtactactgcaccaga
tctccctacggctacgacgagtacggcctggattattggggccagggcaccacagtgaccgtgtcatctggtggaggcggttcaggcgga
ggtggctctggcggtggcggatcggacatccagctgacccagtctccatcctctctgtctgcctctgtgggcgacagagtgaccatgacc
tgcaagtcctctcagtccctgctgaacacccggaaccagaagaactacctgacctggtatcagcagaagcccggcaaggctcccaagctg
ctgatctactgggcctccaccagagaatctggcgtgccctctagattctccggctctggctctggcaccgactttaccctgacaatctcc
agcctgcagcctgaggacttcgccacctactactgccagaacgactacgtgtaccctctgacctttggccagggcaccaagctggaaatc
aagattgaagttatgtatcctcctccttacctagacaatgagaagagcaatggaaccattatccatgtgaaagggaaacacctttgtcca
agtcccctatttcccggaccttctaagcccttttgggtgctggtggtggttgggggagtcctggcttgctatagcttgctagtaacagtg
gcctttattattttctgggtgaggagtaagaggagcaggctcctgcacagtgactacatgaacatgactccccgccgccccgggcccacc
cgcaagcattaccagccctatgccccaccacgcgacttcgcagcctatcgctccagagtgaagttcagcaggagcgcagacgcccccgcg
taccagcagggccagaaccagctctataacgagctcaatctaggacgaagagaggagtacgatgttttggacaagagacgtggccgggac
cctgagatggggggaaagccgagaaggaagaaccctcaggaaggcctgtacaatgaactgcagaaagataagatggcggaggcctacagt
gagattgggatgaaaggcgagcgccggaggggcaaggggcacgatggcctttaccagggtctcagtacagccaccaaggacacctacgac
gcccttcacatgcaggccctgccccctogctaa
Human CD19 CAR:
(SEQ ID NO: 80)
atgcttctcctggtgacaagccttctgctctgtgagttaccacacccagcattcctcctgatcccagacatccagatgacacagactaca
tcctccctgtctgcctctctgggagacagagtcaccatcagttgcagggcaagtcaggacattagtaaatatttaaattggtatcagcag
aaaccagatggaactgttaaactcctgatctaccatacatcaagattacactcaggagtcccatcaaggttcagtggcagtgggtctgga
acagattattctctcaccattagcaacctggagcaagaagatattgccacttacttttgccaacagggtaatacgcttccgtacacgttc
ggaggggggactaagttggaaataacaggctccacctctggatccggcaagcccggatctggcgagggatccaccaagggcgaggtgaaa
ctgcaggagtcaggacctggcctggtggcgccctcacagagcctgtccgtcacatgcactgtctcaggggtctcattacccgactatggt
gtaagctggattcgccagcctccacgaaagggtctggagtggctgggagtaatatggggtagtgaaaccacatactataattcagctctc
aaatccagactgaccatcatcaaggacaactccaagagccaagttttcttaaaaatgaacagtctgcaaactgatgacacagccatttac
tactgtgccaaacattattactacggtogtagctatgctatggactactggggtcaaggaacctcagtcaccgtctcctcagcggccgca
attgaagttatgtatcctcctccttacctagacaatgagaagagcaatggaaccattatccatgtgaaagggaaacacctttgtccaagt
cccctatttcccggaccttctaagcccttttgggtgctggtggtggttgggggagtcctggcttgctatagcttgctagtaacagtggcc
tttattattttctgggtgaggagtaagaggagcaggctcctgcacagtgactacatgaacatgactccccgccgccccgggcccacccgc
aagcattaccagccctatgccccaccacgcgacttcgcagcctatcgctccagagtgaagttcagcaggagegcagacgcccccgcgtac
cagcagggccagaaccagctctataacgagctcaatctaggacgaagagaggagtacgatgttttggacaagagacgtggccgggaccct
gagatggggggaaagccgagaaggaagaaccctcaggaaggcctgtacaatgaactgcagaaagataagatggcggaggcctacagtgag
attgggatgaaaggcgagcgccggaggggcaaggggcacgatggcctttaccagggtctcagtacagccaccaaggacacctacgacgcc
cttcacatgcaggccctgccccctcgctaa

In the sequences above, the underlined part is the scFv sequence, and other structural sequences are as follows:

Nucleic acid sequence of CD28 hinge region
(GenBank: NP_006130.1)
(SEQ ID NO: 81)
attgaagttatgtatcctcctccttacctagacaatgagaagagcaatgg
aaccattatccatgtgaaagggaaacacctttgtccaagtcccctatttc
ccggaccttctaagccc
Nucleic acid sequence of CD28 transmembrane
region
(SEQ ID NO: 82)
ttttgggtgctggtggtggttgggggagtcctggcttgctatagcttgct
agtaacagtggcctttattattttctgggtg
Nucleic acid sequence of CD28 co-stimulatory
signaling region
(SEQ ID NO: 83)
aggagtaagaggagcaggctcctgcacagtgactacatgaacatgactcc
ccgccgccccgggcccacccgcaagcattaccagccctatgccccaccac
gcgacttcgcagcctatcgctcc
CD3ζ sequence (GenBank: BAG36664.1)
(SEQ ID NO: 84)
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtacaatgaactgcagaaagataagat
ggcggaggcctacagtgagattgggatgaaaggcgagcgccggaggggca
aggggcacgatggcctttaccagggtctcagtacagccaccaaggacacc
tacgacgcccttcacatgcaggccctgccccctcgc

The schematic of shRNA structure is shown in FIG. 2, and the sequence is as follows:

(SEQ ID NO: 85)
ACTAGTatcacgagactagcctcgagcggccgcccccttcaccgagggcc
tatttcccatgattccttcatatttgcatatacgatacaaggctgttaga
gagataattggaattaatttgactgtaaacacaaagatattagtacaaaa
tacgtgacgtagaaagtaataatttcttgggtagtttgcagttttaaaat
tatgttttaaaatggactatcatatgcttaccgtaacttgaaagtatttc
gatttcttggctttatatatcttgtggaaaggacgaaacaccggxxxxxx
xxxxxxxxxxxxxxxctcgagxxxxxxxxxxxxxxxxxxxxxtttttgaa
ttctcgacctcgagacaaatggcagtattcatccacaaGGCGCGCC

The xxxx denotes the insertion sites of the seed sequence of shRNA and its complementary sequence.

For the corresponding cell adhesion molecules knocked out by the Crispr method, the crRNA sequences were listed in the table above.

The TracrRNA sequence is as follows:

(SEQ ID NO: 86)
rGrUrUrUrUrArGrArGrCrUrArGrArArArUrArGrCrArArGrUrU
rArArArArUrArArGrGrCrUrArGrUrCrCrGrUrUrArUrCrArArC
rUrUrGrArArArArArGrUrGrGrCrArCrCrGrArGrUrCrGrGrUrG
rCrU*mU*mU*mU

### 1.2 Virus Packaging

The extracted plasmids were packaged with lentiviruses with the specific procedures as follows:
1) The status of 293T cells was confirmed before the experiment, including cell morphology, expansion status, and generations (less than 15);
2) On day one, the 293T cells were seeded in the plate (for T75 flasks, about 6-7×10⁶ cells were seeded in 25 mL of medium, and for T175 flasks, about 15-18×10⁶ cells were seeded in 40 mL of medium);
3) Before virus packaging on day two, when the confluence of 293T cells was greater than 70%-80% and less than 95%, the medium was exchanged with an equal volume of fresh medium, and the culture was continued until the mixture of Tube A and Tube B was ready;
4) Tube A and Tube B were separately prepared (in different volumes for T175 and T75 as described below) and mixed by inversions or low-speed shaking;
T175 system:
Tube A: Opti-MEM (4.6 mL)+Lipo3000 (129 μL)
Tube B: Opti-MEM (4.6 mL)+P3000 (111 μL)+helper plasmids PZ201/PZ202/PZ203 (41.4 μg, 1:1:1 by weight)+CAR molecule plasmid (13.8 μg);
T75 system:
Tube A: Opti-MEM (2 mL)+Lipo3000 (55 μL)
Tube B: Opti-MEM (2 mL)+P3000 (46 μL)+helper plasmids PZ201/PZ202/PZ203 (18 μg, 1:1:1 by weight)+CAR molecule plasmid (6 μg).
5) Tube A was added to Tube B, and the mixture was shaken to mix well and incubated at room temperature for 15 min;
6) The culture flask after the medium exchange in step 3 was inverted such that the medium was in contact with the other side of the culture flask. The Tube A+B mixture was added, and the flask was gently shaken to mix well and then slowly inverted again;
7) The flask was returned to the incubator and incubated for 2 days. The supernatant was collected, centrifuged at 500 g for 10 min, filtered through a 0.45 μm filter membrane, and transferred into a 50-mL centrifuge tube. The tube was sealed and centrifuged at 10000 g at 4° C. for 2 h, and a precipitate was observed;
8) In a biosafety cabinet, the supernatant was discarded, and the centrifuge tube was inversely put on a piece of surgical gauze. After 3 minutes, the inner wall of the tube was gently cleaned with another piece of gauze without touching the precipitate;
9) The precipitate was dissolved in 200 μL of X-vivo medium. 2 μL of the solution was taken for titer determination. The remaining solution was labeled with the LV virus ID, batch number, operator, and packaging volume and then preserved at −80° C.

### EXAMPLE 2: PREPARATION OF CAR-T-PERICRUISER® CELLS (TARGETING HUMAN CD18, CD11A, CD29, AND CD49D) BY SHRNA OR CRISPR METHODS

### 2.1 For the shRNA Method, the Specific shRNA Molecules were: shCD18-3, shCD11a-1, shCD29-3, and shCD49a-2

Human peripheral blood mononuclear cells (PBMCs) were cultured in a medium (X-VIVO15 medium containing 5% of fetal bovine serum+100 U/mL of penicillin +0.1 mg/mL of streptomycin, and 300 IU/mL of IL-2). T cells were activated with the CD2/CD3/CD28 T cell activation and expansion kit (Miltenyi Biotec). Magnetic beads were mixed with the cells in a ratio of 1:2, and the cell density was adjusted to 5×10⁶ cells/mL/cm². After mixing, the cells were cultured at 37° C. and 5% CO₂ for 48 h;

RetroNectin (Takara) was diluted to 20 μg/mL and transferred to a plate (non-tissue culture treated) plate at 4 μg/cm². The plate was incubated in a refrigerator at 4° C. overnight;

After 48 h of activation, the T cells were centrifuged at 300 g for 5 min. The supernatant was discarded, and the cells were then re-suspended in a fresh medium, and transferred to the RetroNectin-coated plate before the CAR-expressing lentivirus was added (MOI=5). The mixture was incubated at 37° C. in 5% CO₂ incubator;

T cells without CAR-expressing lentivirus were prepared in parallel (abbreviated as UNT);

At 24 h after the addition of the lentivirus, the mixture was centrifuged at 300 g for 5 min. The supernatant was discarded, and the T cells were re-suspended to give CAR-T cells (abbreviated as CAR-T-Pericruiser®);

### 2.2 For the Crispr Method, the Specific Molecules were crRNA-CD29-2 and crRNA-CD49-3

The procedures for viral CAR vector infection were similar to those above. The sgRNA (crRNA+tracrRNA) of related cell adhesion molecules was transferred by electroporation (EP) two days after T cells were infected with the CAR vector as follows:

An electroporation medium X-VIVO15+5% FBS+IL2 (antibiotic-free) was prepared;

The medium was added to the plate and pre-heated;
1) The sgRNA was prepared with DNase-free water (1.5 nmol in 10 μL of H₂O), and the cas9 protein was thawed and put in an ice bath for later use;
2) The number of cells for electroporation was calculated as 1E6/run;
3) The desired number of cells were taken and centrifuged to completely remove the medium;
4) The electroporation buffer was prepared during the centrifugation at 26 μL/run according to the calculated volume. Taking 100 μL as an example, 50 μL of buffer A+50 μL of buffer B were taken and mixed in a ratio of 1:1;
5) A proper amount of cells were re-suspended in the electroporation buffer at 20 μL, and the suspension was dispensed into EP tubes at 20 mL/tube;
6) Preparation of RNP: 5 μL of electroporation buffer+0.5 μL of sgRNA+0.5 μL of cas9 protein, and the mixture was well mixed and let stand for 5-10 minutes;
7) The electroporation system was set to 560 V before the test;
8) 20 μL of cells were added to RNP before the electroporation, and the mixture was well mixed and added to the electroporation cuvette with the pipette tip reaching the bottom of the cuvette to avoid air bubbles until a small protrusion was observed at the liquid level. The electroporation cuvette was capped and the electroporation was started;
9) After the completion of the electroporation, the electroporation cuvette was taken out and the cells were transferred to a culture medium;
10) The cells were detected 5 days after the electroporation.

### 2.3 Maintenance of CAR Molecule Expression on Surface of CAR-T-Pericruiser® Cells

48 h after the lentivirus was added to the T cells, UNT and EpCAM-CAR-T-Pericruiser® cells were re-suspended using a flow cytometry washing buffer (PBS+2% of fetal bovine serum), wherein the EpCAM-FITC antibody used was synthesized by Biointron. The mixture was incubated in a 4° C. refrigerator for half an hour for staining and re-suspended in the flow cytometry washing buffer. The transduction rate was detected on a Beckman flow cytometer.

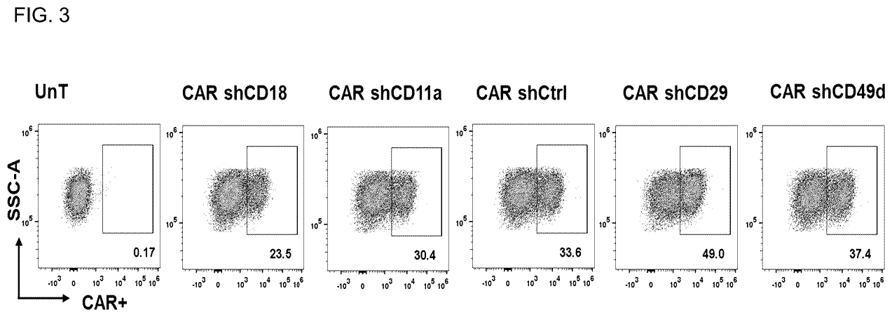

The results of flow cytometry are shown in FIG. 3. The results showed that after CD18, CD11a, CD29, or CD49d was knocked down by the lentivirus vector (expressing the shRNA of the corresponding adhesion-associated molecule) in the EpCAM-CAR-T-Pericruiser® cells, the expression of CAR molecules on the surface of CAR-T cells was retained.

### 2.4 Down-Regulation of Related Cell Adhesion Molecules in EpCAM-CAR-T-Pericruiser® Cells

The CAR-T cells were transfected with the shRNA or Crispr vector for relevant candidate molecules, collected, and re-suspended in a flow cytometry washing buffer (PBS+2% of fetal bovine serum). Each cell was divided into 3 groups, group 1 is blank control with no staininng, anti-CD18-PE, anti-CD29-PE, anti-CD11a-PE, or anti-49d-PE (Biolegend) antibody was added in group 2, and the corresponding Isotype was added in group 3. The mixture was incubated in a 4° C. refrigerator for half an hour for staining and re-suspended in the flow cytometry washing buffer. The detection was conducted on a Beckman flow cytometry.

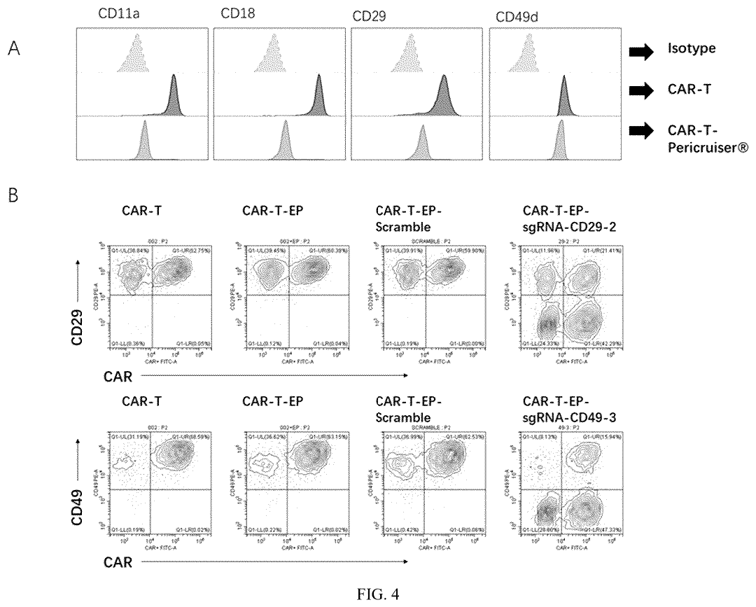

The assay results are shown in FIG. 4: CD18, CD11a, CD29, and CD49d in the EpCAM-CAR-T-Pericruiser® cells were down-regulated correspondingly, with levels higher than that of the Isotype group (UNT) but lower than that of the transduction CAR vector group. FIG. 4A shows the knockdown of the corresponding molecules by the shRNA method and FIG. 4B shows the knockout of the corresponding molecules by the Crispr gene editing method.

### 2.5 CAR-T-Pericruiser® Cells with Decreased Cell Adhesion Capability

24-well culture plates were coated 12 h in advance with a 10 μg/mL working solution of ICAM-1 and VCAM-1 proteins at 100 μL/well. On the next day, the coating solution was discarded, and the plate was washed with 1 mL of PBS once before, then a 5% BSA solution was added to block the plate for 30 min. The 5% BSA solution was then discarded, and the plate was washed with 1 mL of PBS once and preserved for later use.

CAR-T-Pericruiser® cells and UNT cells were collected, washed, re-suspended in PBS, stained with CFSE at 37° C. for 15 min, re-suspended, and washed. PMA group: The CAR-T-Pericruiser® and UNT cells were stimulated with 10 ng/mL PMA for 30 min, re-suspended, and washed. PMA+VLA-4 group: After PMA treatment, a 10 μg/mL anti-VLA-4 blocking antibody was added to each well of the pre-coated plates. The plates were incubated for 15 min. The cells were collected, and the non-adhered CAR-T and UNT cells were gently washed thrice with 1 mL of PBS, with the pipette tip not in contact with the cells on the bottom of the plates. The number of cells collected was detected on a Beckman flow cytometry.

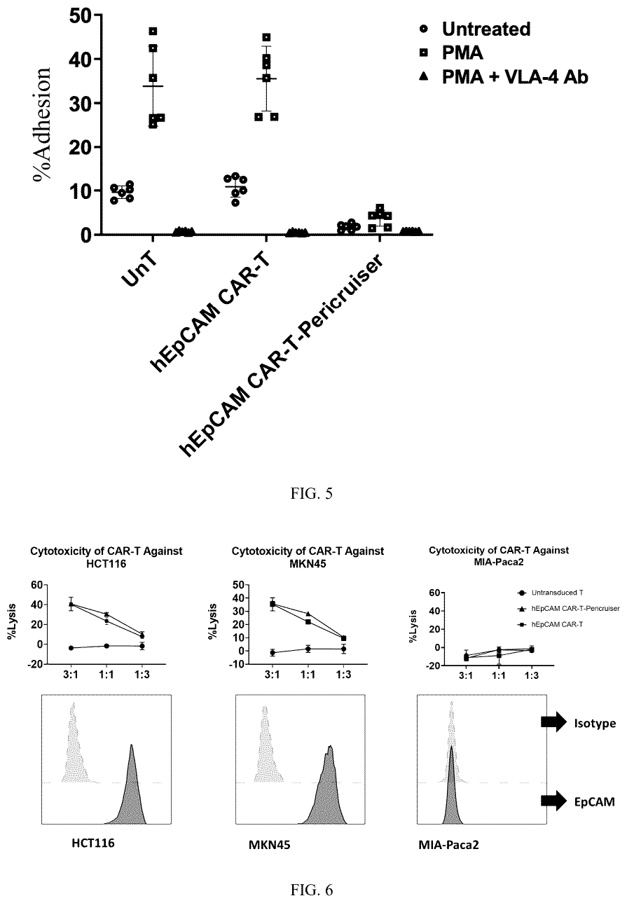

The results are shown in FIG. 5. The adhesion capability of UNT cells and CAR-T cells was greatly up-regulated when PMA was added, and was greatly reduced to nearly absent after VLA-4 Ab was added. The adhesion capability of CAR-T-Pericruiser® cells was very low or nearly absent when PMA and VLA-4 Ab were added.

### EXAMPLE 3: T CELLS EXPRESSING CAR (TARGETING HUMAN EPCAM) MOLECULES EXHIBITED KILLING EFFECTS ON TARGET CELLS CONTAINING CORRESPONDING ANTIGENS (BY SHRNA METHOD)

In 96-well plates, the prepared CAR-T-Pericruiser® (shRNA-CD29-3) and UNT cells were separately co-incubated with 1×10⁴ tumor cells (HCT116, MKN45, or MIA-Paca2) in different effector-to-target ratios (3:1, 1:1, or 1:3) in triplicate in a 5% CO₂ incubator at 37° C. overnight. The release of LDH from target cells was detected by using an LDH kit (Sigma), and the killing efficiency was calculated from the absorbance at 490 nm and 620 nm on a microplate reader;

As shown in FIG. 6, EpCAM-CAR-T-Pericruiser® exhibited dose-dependent killing effects on HCT116 and MKN45 cells and low or no killing effects on MIA-Paca2 cells.

EXAMPLE 4: MOUSE EPCAM-CAR-T-PERICRUISER® CELLS (MEPCAM-CAR-T-CD29SHRNA; BY SHRNA METHOD) TREATED M-NSG XENOGRAFT TUMORS WITH GOOD DOSE-DEPENDENCE AND SAFETY

Mouse breast cancer cells 4T1 were grafted subcutaneously in M-NSG mice at an amount of 1E6 cells. 7 days later. EpCAM-CAR-T and EpCAM-CAR-T-Pericruiser® cells at 0.4 M, 1.5 M and 6 M, and untransduced T (UNT) cells at 6 M were administered via the tail vein. Acute toxicity was observed in all mice in the 6 M EpCAM-CAR-T cell group upon the infusion, and all of the mice died in about two weeks. No acute toxicity was observed in all treatment groups receiving EpCAM-CAR-T-Pericruiser® cells and untransduced T cells, suggesting that EpCAM-CAR-T-Pericruiser® cells have good safety performance.

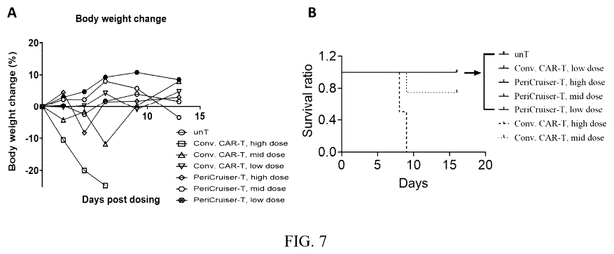

As shown in FIG. 7, the mice in the high-dose 6 M EpCAM-CAR-T group experienced acute toxicity since the start of the cell infusion and weight loss until death by about 2 weeks, while such toxicities were not observed in the EpCAM-CAR-T-Pericruiser® cell and UNT cell groups.

### EXAMPLE 5: HIGH-DOSE HUMAN EPCAM-CAR-T-PERICRUISER® CELLS (BY SHRNA METHOD) EXHIBITED NO SIGNIFICANT TOXICITY IN MOUSE TREATMENT MODEL

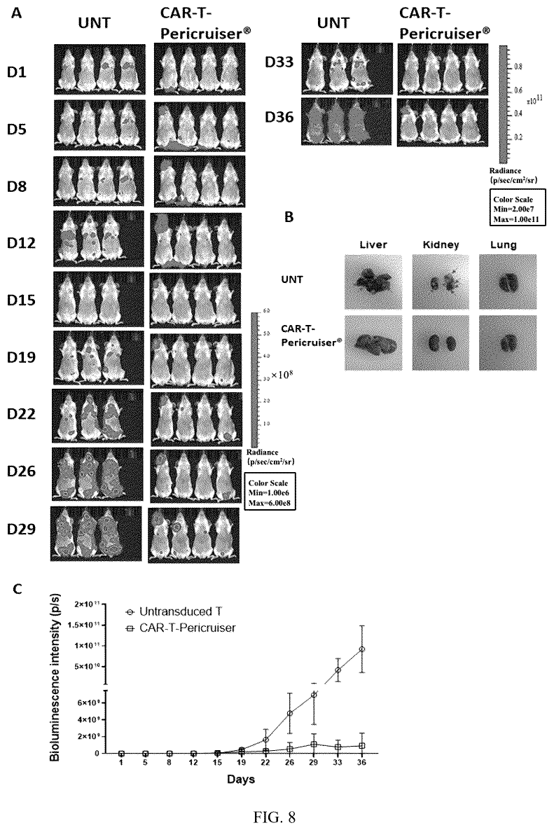

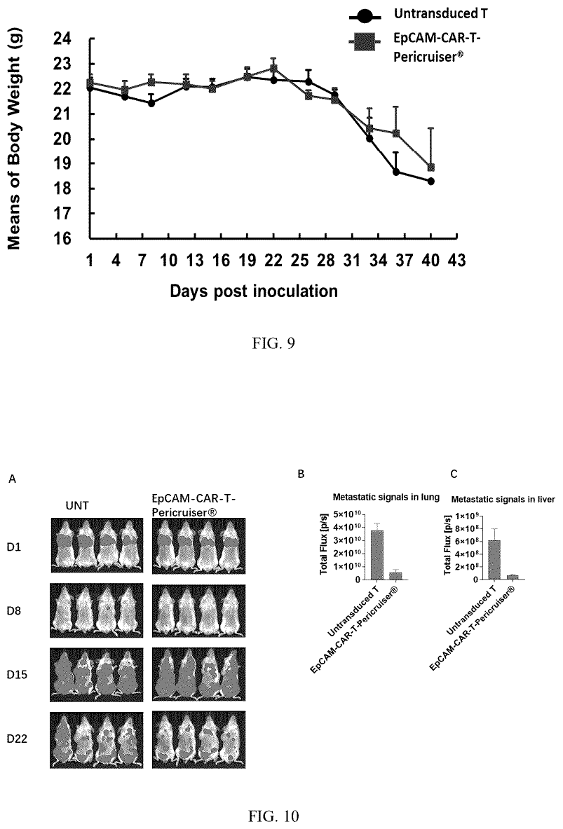

10E6 UNT (untransduced T) cells or EpCAM-CAR-T-Pericruiser® (shRNA-CD29-3) cells were grafted in M-NSG mice, and 4 hours later. 5E5 HCT116-Luc cells were administered via the tail vein. The mice were observed over 40 days. The results showed that EpCAM-CAR-T-Pericruiser® cells are effective in controlling the tumorigenesis of HCT116 cells, with a signal intensity of the tumor fluorescence imaging significantly lower than that of the UNT group (FIGS. 8A-8C); no acute toxicity was seen in mice over 40 days in mice receiving a high dose of EpCAM-CAR-T-Pericruiser® cells (10E6/mouse) with no significant changes in body weight compared with the UNT group (FIG. 9). There is no transgenic hEpCAM mouse available for comprehensive in vivo safety validation. The results in this section and the results of the alternative in vivo study of mouse EpCAM-CAR-T-Pericruiser® cells in NSG mice above suggest that the design of cell adhesion molecule down-regulation can improve the safety of conventional second-generation CAR-T cells in in vivo applications.

### EXAMPLE 6: HUMAN EPCAM-CAR-T-PERICRUISER® CELLS (BY SHRNA METHOD) EFFECTIVELY CONTROLLED TUMORS IN SITU AND METASTASES

1E6 HCT116 tumor cells were grafted subcutaneously in M-NSG mice, and 7 days later, another 5E5 HCT116 cells were grafted again via the tail vein. EpCAM-CAR-T-Pericruiser® (shRNA-CD29-3) cells or untransduced T cells were administered at 4 M 4 hours later. The results, as shown in FIG. 10, suggest that EpCAM-CAR-T-Pericruiser® cells can well control the tumor (FIG. 10A), and the signal of tumor metastasis foci in lung and liver tissues in mice receiving EpCAM-CAR-T-Pericruiser® cells was weaker compared with that in the UNT group at the end of study (FIGS. 10B and 10C).

### EXAMPLE 7: HUMAN EPCAM-CAR-T-PERICRUISER® CELLS (BY SHRNA METHOD) EFFECTIVELY CONTROLLED TAIL VEIN METASTASES

5E5 HCT116 tumor cells were grafted in M-NSG mice via the tail vein, and EpCAM-CAR-T-Pericruiser® (shRNA-CD29-3) cells or untransduced T cells were administered at 10 M 4 hours later. The results showed that EpCAM-CAR-T-Pericruiser® cells can well control the tumor, and the tumor metastatic foci in lung and liver tissues of mice in the EpCAM-CAR-T-Pericruiser® cell group were significantly less than those in the UNT group at the end of study (FIG. 8B).

### EXAMPLE 8: HUMAN EPCAM-CAR-T-PERICRUISER® CELLS (BY SHRNA METHOD) IN COMBINATION WITH CHEMOTHERAPY EFFECTIVELY CONTROLLED ABDOMINAL METASTASES

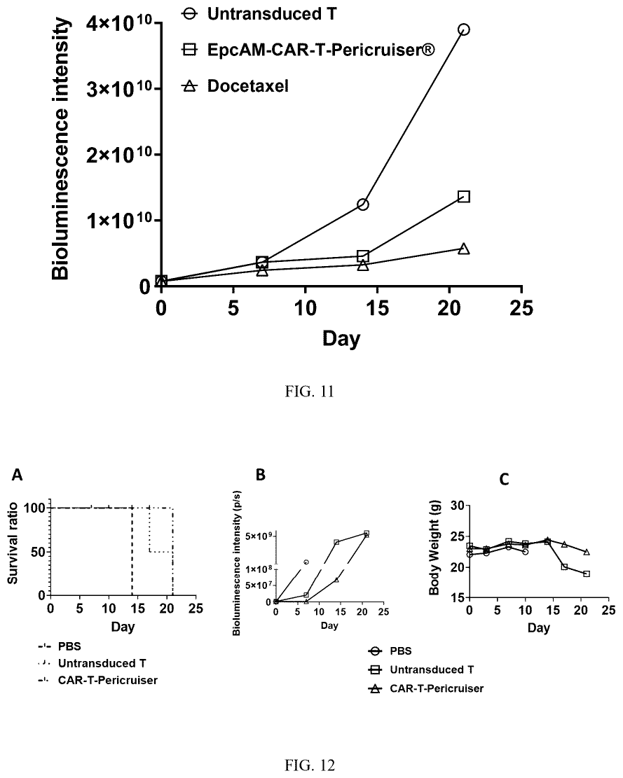

1E6 MKN45 cells were grafted intraperitoneally in M-NSG mice, and 7 days later, 4E6 untransduced T cells. EpCAM-CAR-T-Pericruiser® (shRNA-CD29-3) cells, chemotherapeutic agent docetaxel (4.5 mg/kg), and EpCAM-CAR-T-Pericruiser® cells+chemotherapeutic agent docetaxel (4.5 mg/kg) were administered via the tail vein. The results show that the combination of EpCAM-CAR-T-Pericruiser® cells with chemotherapy can effectively control abdominal tumor metastases with better efficacy than those of the CAR-T cell group or chemotherapeutic agent group (FIG. 11).

### EXAMPLE 9: HUMAN CD19 CAR-T-PERICRUISER® CELLS (BY SHRNA METHOD) EFFECTIVELY CONTROLLED TAIL VEIN METASTASES OF NALM-6-LUC TUMOR IN NSG MICE

5E5 NALM-6-Luc cells were injected into NSG mice via the tail vein, and three days later, fluorescence signals of tumors were detected on an in vivo fluorescence imaging system (IVIS) in NSG mice. The mice were randomized into the PBS, UNT cell, and CD19CAR-T-Pericruiser® (shRNA-CD29-3) cell groups. The treatment groups were given 5E6 T cells via the tail vein. The results show that CD19CAR-T-Pericruiser® cells have an antitumor effect relative to PBS and UNT cells without significant toxicity with body weight comparable to that of the PBS group, while the UNT group showed significant weight losses (FIG. 12).

### EXAMPLE 10: MOUSE EPCAM CAR-T IN COMBINATION WITH BLOCKING ANTIBODIES NATALIZUMAB AND EFALIZUMAB SIGNIFICANTLY INHIBITED ON-TARGET OFF-TUMOR TOXICITY OF CAR-T CELLS WITHOUT AFFECTING CAR-T CELL FUNCTIONALITY IN VITRO

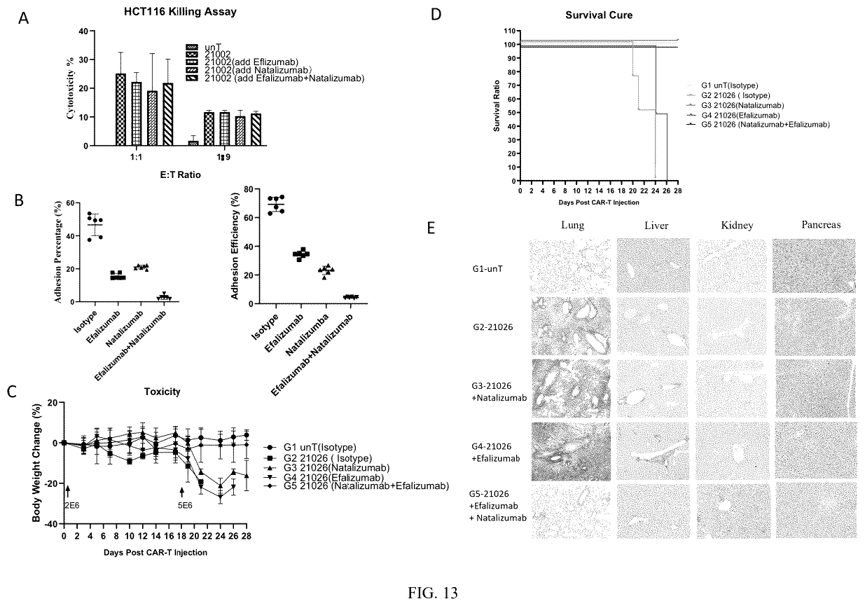

In 96-well plates, the prepared human EpCAM CAR-T (21002) and UNT cells were separately co-incubated with 1×10⁴ tumor cells (HCT116) in different effector-to-target ratios (1:1 or 1:9) in triplicate in a 5% CO₂ incubator at 37° C. overnight. The release of LDH from target cells was detected by using an LDH kit (Sigma), and the killing efficiency was calculated from the absorbance at 490 nm and 620 nm on a microplate reader. The results, as shown in FIG. 13A, suggest that the co-incubation of CAR-T cells with antibodies does not affect the cell killing capacity.

In the protein adhesion assay. ICAM-1 and VCAM-1 proteins were immobilized on 96-well plates at 5 μg/mL. 1E5 CAR-T cells were co-incubated with the antibodies and transferred to the coated plates in triplicate, and the plates were incubated for 3 h at 37° C. in a 5% CO₂ incubator. The non-adhered cells were washed with PBS after the incubation. In the vascular endothelial cell adhesion assay. 1E5 HUVEC cells were activated with 10 ng/mL TNF-a on 96-well plates. 2E5 CAR-T cells were co-incubated with natalizumab or efalizumab and transferred to the plates in triplicate, and the plates were incubated for 6 h at 37° C. in a 5% CO₂ incubator. The non-adhered cells were washed with PBS after the incubation. The results, as shown in FIG. 13B, suggest that the use of efalizumab or natalizumab can effectively inhibit the cell-protein adhesion and reduce the adhesion capability to vascular cells.

In the acute toxicity study, 2E6 and 5E6 mouse EpCAM CAR-T (21026) cells were injected into NSG mice via the tail vein on day 1 and day 7, respectively. 5 mg/kg of natalizumab and 5 mg/kg of efalizumab were administered intraperitoneally in mice twice a week, and the mice were examined for body weight changes after the infusion. T cell infiltration assay was performed in the lungs, liver, kidneys, and pancreas of the mice after death. The results, as shown in FIGS. 13C, 13D, and 13E, suggest that the mouse EpCAM CAR-T cells exhibited significant toxicity with fast weight loss observed in mice; the addition of natalizumab or efalizumab partially reduced the toxicity, and the addition of the two antibodies completely inhibited the on-target off-tumor toxicity of the CAR-T cells with no significant weight loss observed in mice (FIG. 13C); in the CAR-T+antibody combination groups in the acute toxicity study, the survival of mice was significantly prolonged (FIG. 13D); significant CAR-T cell infiltration was observed in the lungs and liver in the CAR-T cell monotherapy group, while no T cell infiltration was seen in lungs, liver, kidneys, and pancreas of the mice in the CAR-T+two antibodies combination group (FIG. 13E).

### EXAMPLE 11: INDIVIDUAL KNOCKOUT OF CELL ADHESION MOLECULES IN T CELLS REDUCED EPCAM CAR-T CELL ADHESION CAPABILITY WITHOUT AFFECTING CAR-T CELL FUNCTIONALITY

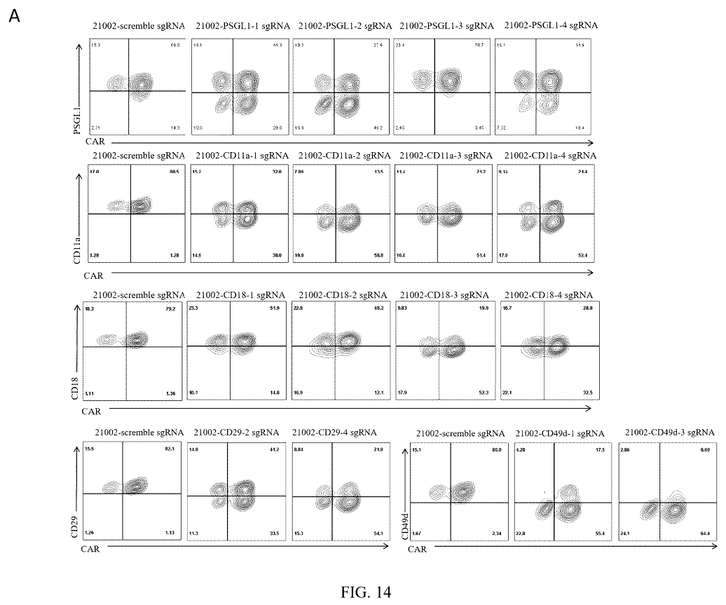

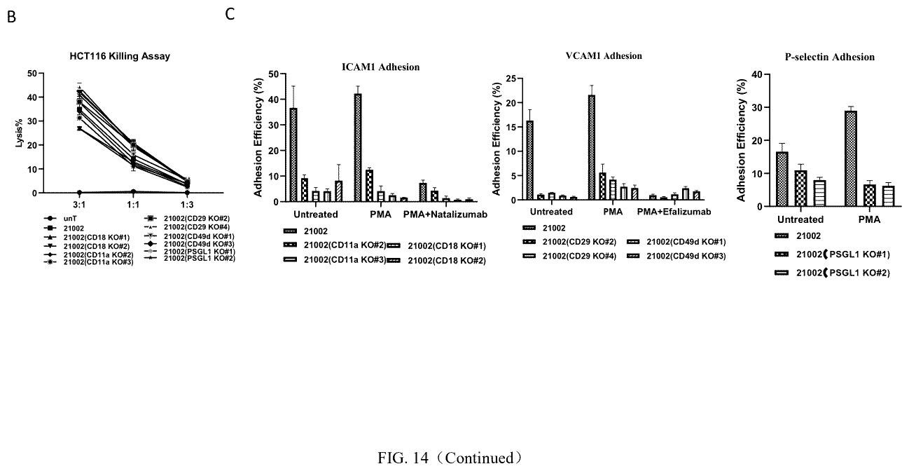

PSGL1, CD11a, CD18, CD29, and CD49d on the membrane surface of EpCAM CAR-T (21002) cells were knocked out by the CRISPR method, and the corresponding sgRNA sequences were: crRNA-PSGL1-2, crRNA-CD11a-3, crRNA-CD18-3, crRNA-CD29-4,and crRNA-CD49d-3, respectively. The sgRNAs and Cas9 protein were introduced into CAR-T cells by electroporation, and the expression of CAR and the target genes was detected by flow cytometric. The results, as shown in FIG. 14A, show that the target proteins can be effectively knocked out, and the expression of CAR molecule is not affected by the knockout of target genes.

In 96-well plates, the CAR-T cells with target gene knockout and the UNT cells were separately co-incubated with 1×10⁴ tumor cells (HCT116) in different effector-to-target ratios (3:1, 1:1, or 1:3) in triplicate in a 5% CO₂ incubator at 37° C. overnight. The release of LDH from target cells was detected by using an LDH kit (Sigma), and the killing efficiency was calculated from the absorbance at 490 nm and 620 nm on a microplate reader. The results, as shown in FIG. 14B, suggest that CAR-T cells with PSGL1, CD11a, CD18, CD29, or CD49d target gene knockout exhibited no difference in the killing capacity against target cells compared with the control group.

In the protein adhesion assay, 5 μg/mL ICAM-1, VCAM-1, and p-selectin were separately immobilized on 96-well plates. 1E5 CAR-T cells were stimulated with PMA, co-incubated with PBS, natalizumab or efalizumab, and transferred to the coated plates in triplicate, and the plates were incubated for 3 h at 37° C. in a 5% CO₂ incubator. The non-adhered cells were washed with PBS after the incubation. The results, as shown in FIG. 14C, suggest that the knockout of CD11a and CD18 can reduce the adhesion of CAR-T cells to ICAM-1, the knockout of CD29 and CD49d can reduce the adhesion of CAR-T cells to VCAM-1, and the knockout of PSGL1 can reduce the adhesion of CAR-T cells to p-selectin.

### EXAMPLE 12: DUAL KNOCKOUT OF CELL ADHESION MOLECULES IN T CELLS REDUCED CAR-T CELL ADHESION CAPABILITY WITHOUT AFFECTING CAR-T CELL FUNCTIONALITY

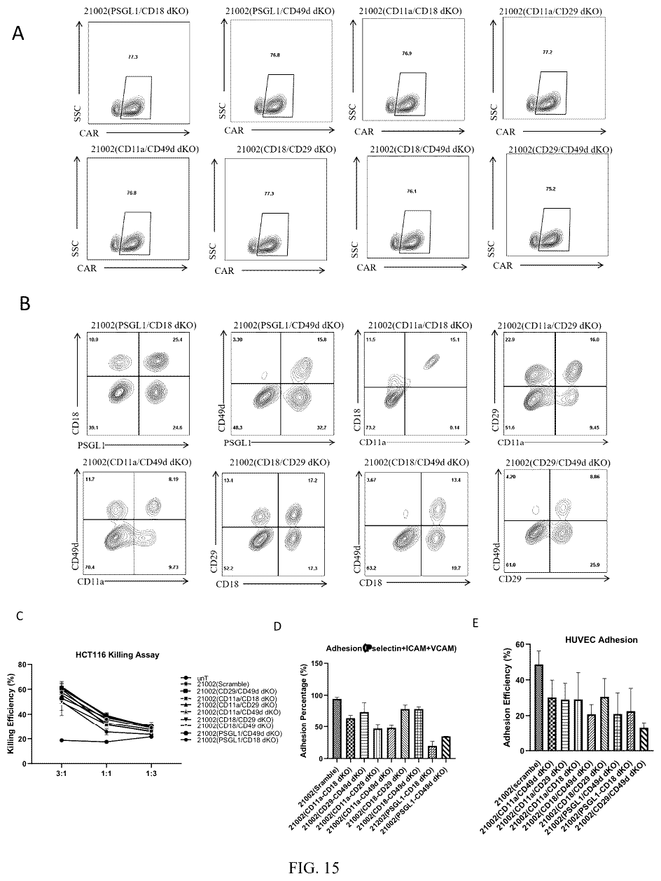

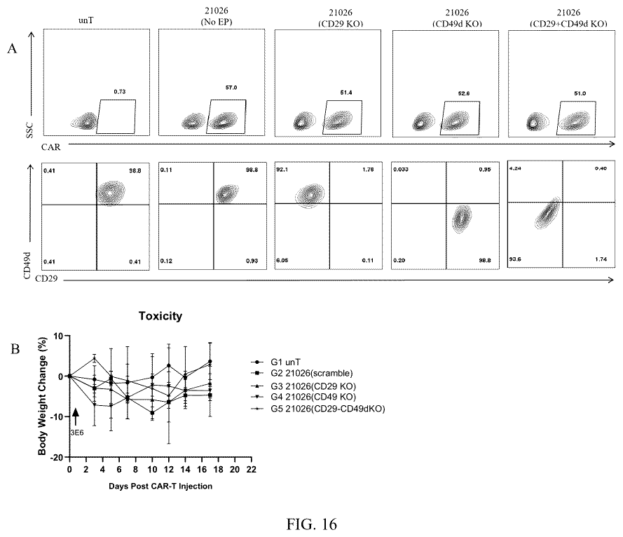

PSGL1, CD11a, CD18, CD29, and CD49d on the membrane surface of EpCAM CAR-T (21002) cells were knocked out by the CRISPR method in combinations of two (PSGL1/CD18; PSGL1/CD49d; CD11a/CD18; CD11a/CD29; CD11a/CD49d; CD18/CD29; CD18/CD49d; and CD29/CD49d), and the corresponding sgRNA sequences were: crRNA-PSGL1-2, crRNA-CD11a-3, crRNA-CD18-3, crRNA-CD29-4, and crRNA-CD49d-3, respectively. As shown in FIG. 15A, the dual knockout combinations above do not affect the CAR expression in CAR-T cells. As shown in FIG. 15B, the dual knockout combinations above can effectively knock out the target genes.

In 96-well plates, the CAR-T cells with dual knockout and the UNT cells were separately co-incubated with 1×10⁴ tumor cells (HCT116) in different effector-to-target ratios (3:1, 1:1, or 1:3) in triplicate in a 5% CO₂ incubator at 37° C. overnight. The release of LDH from target cells was detected by using an LDH kit (Sigma), and the killing efficiency was calculated from the absorbance at 490 nm and 620 nm on a microplate reader. The results, as shown in FIG. 15C, suggest that the knockout combinations above in CAR-T cells do not affect cell killing capacity compared with the control group.

In the protein adhesion assay, ICAM-1, VCAM-1, and p-selectin were separately immobilized on 96-well plates at 5 μg/mL. 1E5 CAR-T cells were transferred to the coated plates in triplicate, and the plates were incubated for 3 h at 37° C. in a 5% CO₂ incubator. The non-adhered cells were washed with PBS after the incubation. The results, as shown in FIG. 15D, suggest that the knockout combinations above in CAR-T cells can reduce the cell adhesion to ICAM-1, VCAM-1, and p-selectin. In the vascular endothelial cell adhesion assay, 1E5 HUVEC cells were activated with 10 ng/ml TNF-a on 96-well plates. 2E5 CAR-T cells were transferred to the plates in triplicate, and the plates were incubated for 6 h at 37° C. in a 5% CO₂ incubator. The non-adhered cells were washed with PBS after the incubation. The results, as shown in FIG. 15E, suggest that the knockout combinations above in CAR-T cells can reduce the adhesion to vascular cells HUVECs.

### EXAMPLE 13: DUAL KNOCKOUT OF CELL ADHESION MOLECULES IN T CELLS REDUCED THE TOXICITY OF MOUSE EPCAM CAR-T CELLS

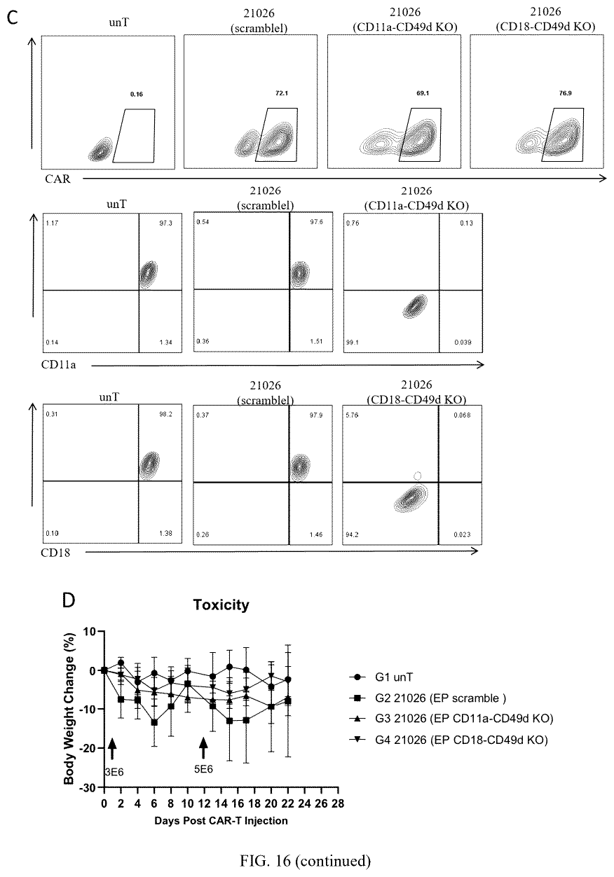

CD29, CD49d, CD29+CD49d, CD18+CD49d, and CD11a+CD49d on the membrane surface of EpCAM CAR-T cells were knocked out by the CRISPR method. As shown in FIGS. 16A and 16C, the dual knockout combinations above do not affect the CAR expression in CAR-T cells; As shown in FIGS. 16A and 16D, the individual knockouts and dual knockout combinations above can effectively knock out the target genes.

3E6 or 5E6 mouse EpCAM CAR-T cells were injected into NSG mice via the tail vein; the mice were monitored for body weight changes after the infusion. The results are shown in FIGS. 16B and 16E. The CAR-T cells with individual knockout of CD29 or CD49d failed to inhibit the on-target off-tumor toxicity and weight loss was observed in the mice when compared with the reference CAR-T cells; the dual knockout combinations CD29+CD49d, CD18+CD49d, and CD11a+CD49d inhibited the on-target off-tumor toxicity and weight loss in mice.

### EXAMPLE 14: INDIVIDUAL KNOCKDOWN OF CELL ADHESION MOLECULES IN T CELLS REDUCED EPCAM CAR-T CELL ADHESION CAPABILITY WITHOUT AFFECTING CAR-T CELL FUNCTIONALITY

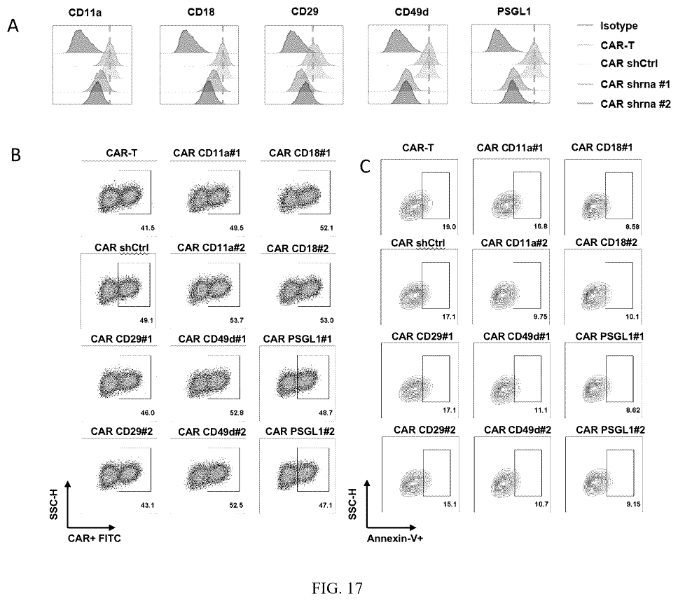

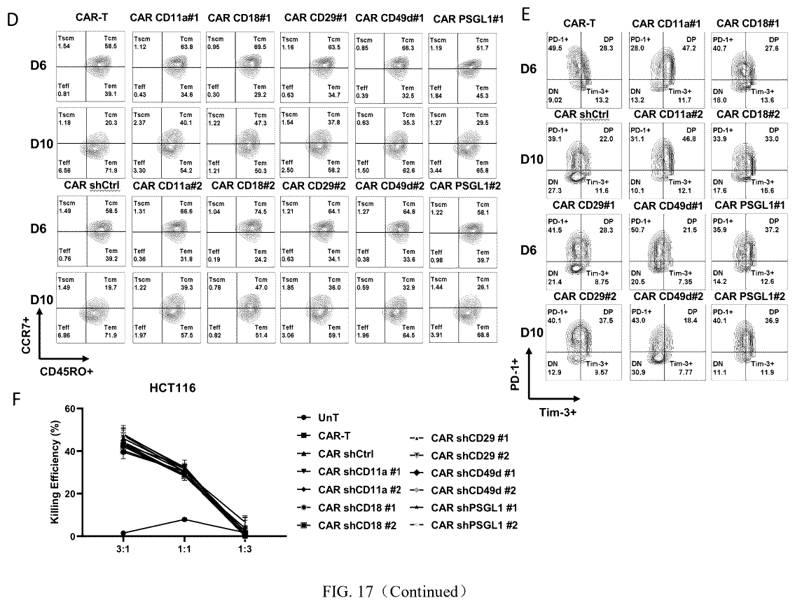

The target genes were knocked down in CAR-positive cells by shRNA co-expressing with CAR. As shown in FIG. 17A, the shRNAs of PSGL1 (#1: shRNA-PSGLI-5; #2: shRNA-PSGL1-7), CD11a (#1: shRNA-CD11a-1; #2: shRNA-CD11a-6), CD18 (#1: shRNA-CD18-10; #2: shRNA-CD18-11), CD29 (#1: shRNA-CD29-3; #2: shRNA-CD29-4), and CD49d (#1: shRNA-CD49d-5; #2: shRNA-CD49d-9) can effectively knock down the target genes; also, as shown in FIG. 17B, the knockdowns of PSGL1, CD11a, CD18,CD29, and CD49d do not affect the expression of CAR on the surface of CAR-T cells; it was found that the knockdown of the target genes can reduce the rate of apoptosis in CAR-T cells as detected by Annexin-V antibody, as shown in FIG. 17C; the memory phenotype of CAR-T cells was detected by flow cytometry using CCR7 and CD45RO antibodies, and as shown in FIG. 17D, the knockdown of PSGL1, CD11a, CD18, CD29, and CD49d can increase the CAR-T cells with memory phenotype; the CAR-T cell exhuasion phenotype was detected by using PD1 antibody and Tim3 antibody, and as shown in FIG. 17E, the knockdown of PSGL1, CD11a, CD18, CD29, and CD49d can inhibit CAR-T cell exhuasion; the CAR-T cells were co-incubated with 4T1 cells or RKO-mEpCAM cells in different effector-to-target ratios, the supernatants were taken after 24 h to detect LDH, then the killing effect of the CAR-T cells on target cells was calculated, and as shown in FIG. 17F, the knockdowns of PSGL1, CD11a, CD18, CD29, and CD49d in CAR-T cells do not affect the killing functionality of CAR-T cells compared with the control group.

### EXAMPLE 15: MULTI-KNOCKDOWN OF CELL ADHESION MOLECULES IN T CELLS EFFECTIVELY REDUCED EPCAM CAR-T CELL ADHESION WITHOUT AFFECTING CAR-T CELL FUNCTIONALITY

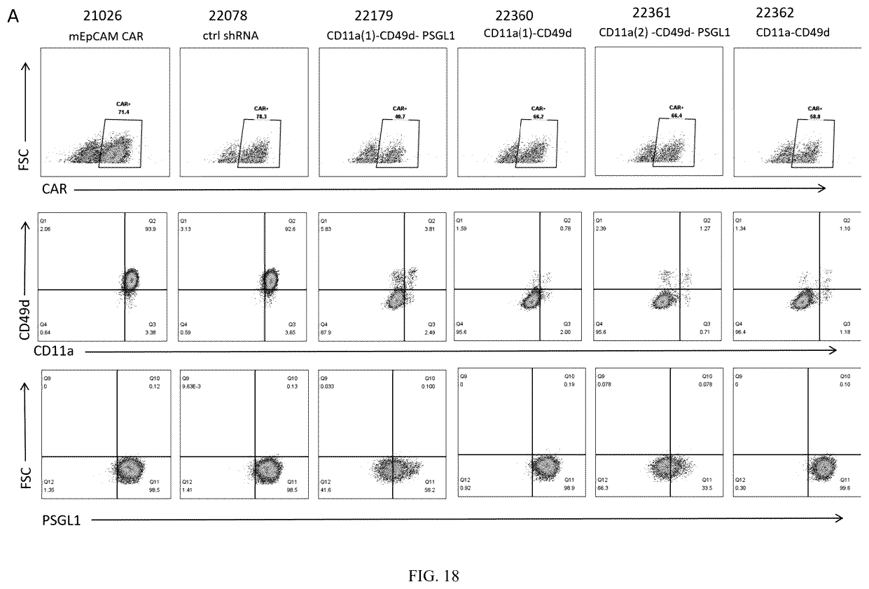

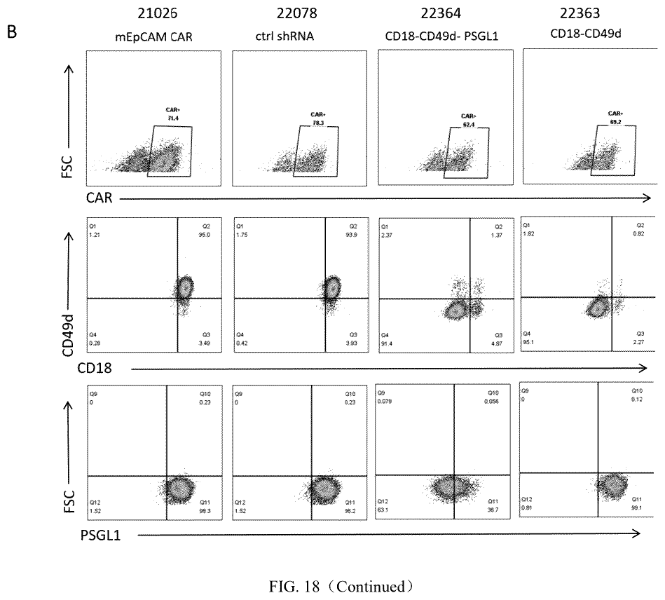

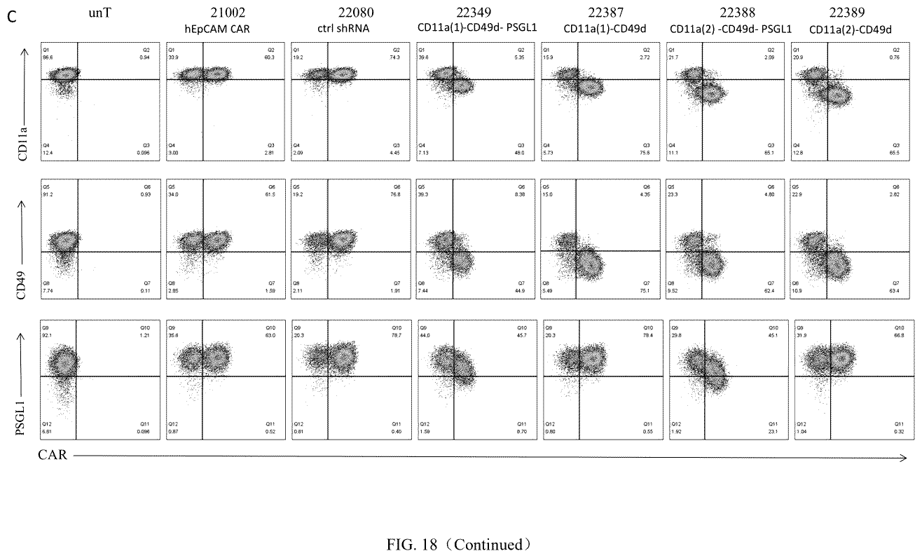

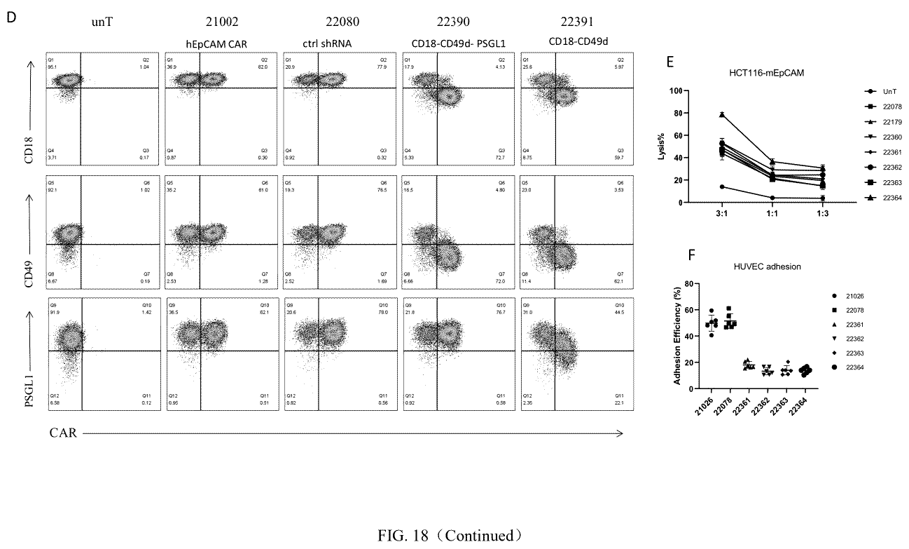

The expression of multiple target molecules in mouse EpCAM CAR-T was simultaneously reduced by multiple shRNAs co-expressing with CAR. As shown in FIG. 18A and 18B, 21026 (mEpCAM CAR-T) and 22078 (ctrl shRNA) were the control groups; 22179 (shRNA-PSGL1-7, shRNA-CD11a-6, shRNA-CD49d-5) and 22361 (shRNA-PSGL1-7, shRNA-CD11a-9, shRNA-CD49d-5) specifically knocked down PSGL1, CD11a, and CD49d; 22360) (shRNA-CD11a-6, shRNA-CD49d-5) and 22362 (shRNA-CD11a-9, shRNA-CD49d-5) specifically knocked down CD11a and CD49d; 22363 (shRNA-CD18-14, shRNA-CD49d-5) specifically knocked down CD18 and CD49d; 22364 (shRNA-PSGL1-7, shRNA-CD18-14, shRNA-CD49d-5) specifically knocked down PSGL1, CD18, and CD49d. The flow cytometry results show that the molecules above can effectively reduce the expression of the target genes without affecting the CAR expression.

The expression of multiple target genes in human EpCAM CAR-T was simultaneously reduced by the multi-shRNA method. As shown in FIG. 18C and 18D, 21002 (hEpCAM CAR-T) and 22080 (ctrl shRNA) were the control groups; 22349 (shRNA-PSGL1-7, shRNA-CD11a-6, shRNA-CD49d-5) and 22388 (shRNA-PSGL1-7, shRNA-CD11a-9, shRNA-CD49d-5) specifically knocked down PSGL1, CD11a, and CD49d; 22387 (shRNA-CD11a-6, shRNA-CD49d-5) and 22389 (shRNA-CD11a-9, shRNA-CD49d-5) specifically knocked down CD11a and CD49d; 22390 (shRNA-CD18-14, shRNA-CD49d-5) specifically knocked down CD18 and CD49d; 22391 (shRNA-PSGL1-7, shRNA-CD18-14, shRNA-CD49d-5) specifically knocked down PSGL1, CD18, and CD49d. The flow cytometry results show that the molecules above can effectively knock down the target genes without affecting the CAR expression.

In 96-well plates, the 22078, 22179, 22360, 22361, 22362, 22363, and 22364 CAR-T cells and the UNT cells were separately co-incubated with 1×10⁴ tumor cells (HCT116) in different effector-to-target ratios (3:1, 1:1, or 1:3) in triplicate in a 5% CO₂ incubator at 37° C. overnight. The release of LDH from target cells was detected by using an LDH kit (Sigma), and the killing efficiency was calculated from the absorbance at 490 nm and 620 nm on a microplate reader. The results, as shown in FIG. 18E, suggest that the knockout combinations above in CAR-T cells do not affect cell killing capacity compared with the control group.

In the vascular endothelial cell adhesion assay, 1E5 HUVEC cells were activated with 10 ng/mL TNF-a on 96-well plates. 2E5 CAR-T cells were transferred to the plates in triplicate, and the plates were incubated for 6 h at 37° C. in a 5% CO₂ incubator. The non- adhered cells were washed with PBS after the incubation. The results, as shown in FIG. 18F, suggest that the knockdown combinations above in CAR-T cells can inhibit the cell adhesion to vascular cells HUVECs.

### EXAMPLE 16: MULTI-KNOCKDOWN OF CELL ADHESION MOLECULES IN T CELLS EFFECTIVELY REDUCED THE IN VIVO TOXICITY OF MOUSE EPCAM CAR-T CELLS

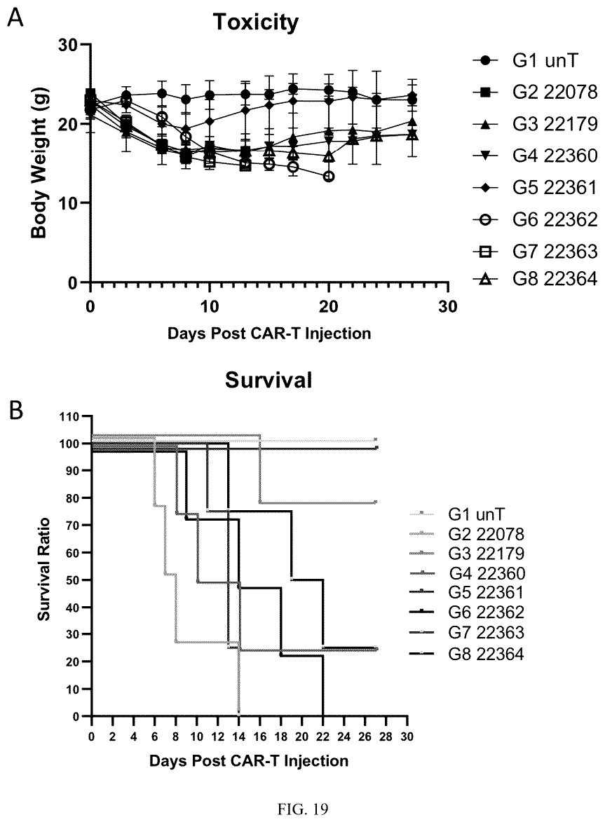

5E6 22078, 22179, 22360, 22361, 22362, and 22363 mouse EpCAM CAR-T cells were injected into NSG mice via the tail vein; the mice were monitored for body weight changes after the infusion. The results are shown in FIG. 19A. The weight of mice in the 22078 group was reduced significantly, and death was observed; mice in the 22179 and 22361 groups exhibited mild weight loss, which soon returned to normal; partial improvement in weight of mice was also found in the other groups compared with that in the 21078 group. The survivals of the groups are shown in FIG. 19B. 22179 and 22361 significantly prolonged the survival in the mice.

### EXAMPLE 17: MULTI-KNOCKDOWN OF CELL ADHESION MOLECULES IN T CELLS RETAINED THE ABILITY OF CAR-T CELLS TO ELIMINATE METASTASIS TUMORS AND SOLID TUMORS

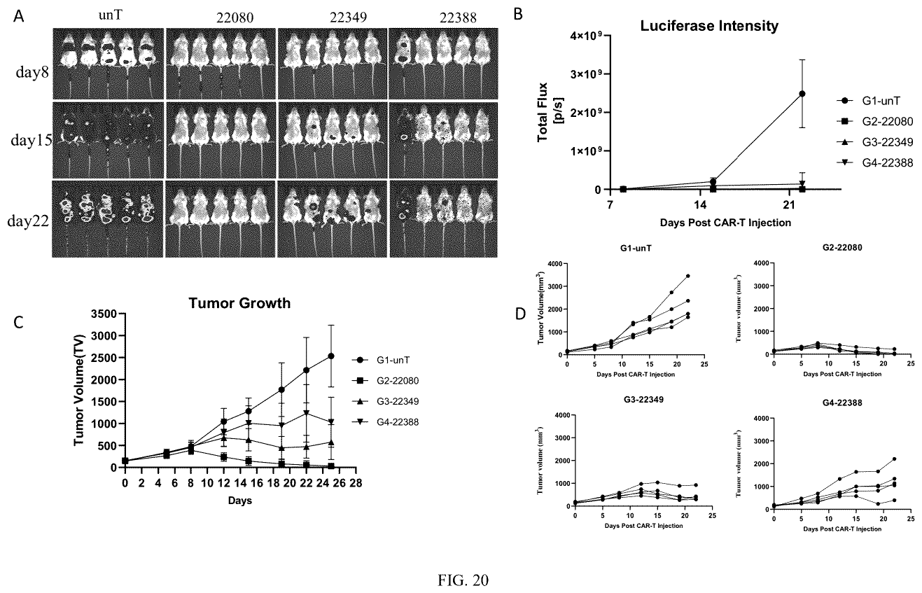

5E6 HCT116 cells were subcutaneously injected into NSG mice. When the tumor grew to 150 mm³, 5E6 CAR-T cells were intravenously injected, and on the day after the CAR-T administration, 2E6 HCT116-Luc cells were intravenously injected. The subcutaneous tumors were detected periodically, and the tumor metastases were detected by in vivo imaging. As shown in FIGS. 20A and 20B, 22080, 22349, and 22388 can effectively eeliminate metastatic HCT116-luc tumor cells with no significant difference among the groups. As shown in FIGS. 20C and 20D, both 22080 and 22349 can effectively inhibit the growth of subcutaneous HCT116 solid tumors, while 22388 exhibited slightly reduced inhibitory effects.

The above examples only illustrate several embodiments of the present disclosure for the purpose of specific and detailed description, but should not be construed as limiting the scope of the present disclosure. It should be noted that various modifications and improvements can be made by those of ordinary skill in the art without departing from the spirit of the present disclosure, and such modifications and improvements shall fall within the scope of the present disclosure. Therefore, the protection scope of the present invention is determined by the claims, and the specification and the accompanying drawings can be used to illustrate the content of the claims.

1. An immune cell for use as a tumor killer cell in an adoptive immune cell therapy with down-regulated cell adhesion capability.

2. The immune cell of claim 1, wherein the immune cell comprises a cell adhesion molecule with reduced expression or inhibited functionality.

3. The immune cell of claim 2, wherein the cell adhesion molecule comprises one or more selected from the group consisting of IgSF CAM, integrin, cadherin, selectin, and chemokine receptor.

4. The immune cell of claim 3, wherein the cell adhesion molecule comprises one, two or more selected from the group consisting of PSGL-1, CD44, CD11a, CD18, CD49d, CD29, and CXCR4.

5. The immune cell of claim 2, wherein at least part of the cell adhesion molecules are knocked down or knocked out, or blocked by an antibody.

6. The immune cell of claim 1, wherein the adoptive immune cell therapy is an NK therapy, a LAK therapy, a DC therapy, a CIK therapy, a TIL therapy, a DC-CIK therapy, a CAR-T therapy, a TCR-T therapy, a CAR-NK therapy, or a TCR-NK therapy.

7. The immune cell of claim 6, wherein the immune cell expresses a chimeric antigen receptor; the chimeric antigen receptor comprises an extracellular antigen-recognition domain for recognizing a tumor antigen, a hinge region, a transmembrane domain, and an intracellular signaling domain; and the antigen-recognition domain specifically recognizes and binds to at least one of following antigen molecules:
alpha-fetoprotein, alpha-actinin-4, A3, an antigen specific to an A33 antibody, ART-4, B7, B7H3, Ba 733, BAFF-R, BAGE, BCMA, BrE3 antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD56, CD59, CD64, CD66a/b/c/c, CD67, CD70, CD70L, CD74, CD79a, CD79b, CD80, CD83, CD95, CD117, CD123, CD126, CD132, CD133, CD138, CD147, CD154, CD319, CD371, CDC27, CDK-4/m, CDKN2A, CLL1, CTLA4, CXCR4, CXCR7, CXCL12, HIF-1α, colon-specific antigen-p (CSAp), CEA, CEACAM-6, c-Met, DAM, EGFR, EGFRvIII, EGP-1, EGP-2, ELF2-M, EpCAM, fibroblast growth factor, Flt-1, Flt-3, folate receptor, G250 antigen, GAGE, gp100, GRO-β, GPRC5D, HLA-DR, HM1.24, human chorionic gonadotropin (HCG) and a subunit thereof, HER2/neu, HMGB-1, hypoxia-inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor 1, Igκ, IL1RAP, Lewis Y, LMP1, KC4 antigen, KS-1 antigen, KS1-4, Le-Y, LDR/FUT, macrophage migration inhibitory factor, MAGE, MAGE-3, MART1, MART-2, NY-ESO-1, TRAG-3, mCRP, MCP-1, MMG49, MIP-1A, MIP-1B, MIF, MUC1, MUC2, MUC3, MUC4, MUC5ac, MUC13, MUC16, MUM-1/2, MUM-3, NCA66, NCA95, NCA90, NKG2D ligand, pancreatic mucin, PD-L1, placental growth factor, p53, PLAGL2, prostatic acid phosphatase, PSA, PRAME, PSMA, PlGF, ILGF, ILGF-1R, IL-6, IL-25, RS5, RANTES, ROR1, T101, SAGE, S100, survivin, TAC, TAG-72, tenascin, TRAIL receptor, TNF-alpha, Tn antigen, Thomsen-Friedenreich antigen, tumor necrosis antigen, VEGFR, ED-B fibronectin, WT-1, 17-1A antigen, complement components C3, C3a, C3b, C5a and C5, angiogenesis markers, bc1-2, bc1-6, Kras, oncogenic markers and oncogenic gene products.

8. The immune cell of claim 7, wherein the antigen-recognition domain is scFv.

9. The immune cell of claim 1, wherein the immune cell is a T cell, a B cell, an NK cell, or a DC cell.

10. The immune cell of claim 9, wherein the T cell is selected from the group consisting of a helper T cell, a cytotoxic T cell, a memory T cell, a regulatory T cell, a MAIT cell, and a γδT cell.

11. A nucleic acid construct, comprising a first expression cassette and a second expression cassette, wherein
the first expression cassette is used to express an antagonistic component of a cell adhesion molecule;
the second expression cassette is used to express a chimeric antigen receptor;
the antagonistic component is a molecule that can specifically inhibit transcription or translation of the cell adhesion molecule, or can specifically inhibit expression or activity of a protein of the cell adhesion molecule;
the first expression cassette and the second expression cassette are located on a same nucleic acid construct, or on different nucleic acid constructs; and
the cell adhesion molecule has reduced expression or inhibited functionality, and the chimeric antigen receptor is defined as in claim 7.

12. The nucleic acid construct of claim 11, wherein the antagonistic component is selected from the group consisting of a nucleic acid molecule, an antibody drug, and an interfering lentivirus.

13. The nucleic acid construct of claim 12, wherein the nucleic acid molecule is selected from the group consisting of an antisense oligonucleotide, dsRNA, microRNA, siRNA, shRNA, and a nucleic acid encoding CRISPR system.

14. A method for preparing an immune cell for use as a tumor killer cell in an adoptive immune cell therapy with down-regulated cell adhesion capability, comprising: transferring the nucleic acid construct of claim 11 into an immune cell for expression; or
treating an immune cell with the antagonistic component described in claim 11, and transferring the second expression cassette into the immune cell before, after, or during the treatment.

15. A pharmaceutical composition comprising the immune cell of claim 1.

16. A pharmaceutical combination product or pharmaceutical composition comprising a tumor killer cell for use in an adoptive immune cell therapy and an antagonistic component, wherein
the antagonistic component can induce reduced expression or inhibited functionality of a cell adhesion molecule on a surface of the tumor killer cell to obtain the immune cell of claim 1.

17. The pharmaceutical combination product or pharmaceutical composition of claim 16, wherein the antagonistic component is selected from the group consisting of a nucleic acid molecule, an antibody drug, and an interfering lentivirus.

18. The pharmaceutical combination product or pharmaceutical composition of claim 17, wherein the nucleic acid molecule is selected from the group consisting of an antisense oligonucleotide, dsRNA, microRNA, siRNA, shRNA, and a nucleic acid encoding CRISPR system.

19. A method of preventing and/or treating a tumor in a subject, comprising administering to the subject the immune cell of claim 1.

20. A method of preventing and/or treating a tumor in a subject, comprising administering to the subject a tumor killer cell for an adoptive immune cell therapy in combination with an antagonistic component, wherein
the tumor killer cell for the adoptive immune cell therapy and the antagonistic component are defined as in claim 16.

21. A method for preventing and/or treating a tumor, comprising administering to a subject an effective amount of a pharmaceutical composition comprising an immune cell for use as a tumor killer cell in an adoptive immune cell therapy with down-regulated cell adhesion capability or the pharmaceutical combination product or pharmaceutical composition of claim 16.

22. The method of claim 21, wherein the method is used to treat an in situ lesion and/or metastasis of the tumor.

23. The method of claim 21, wherein the method is used to reduce in vivo toxicity of the tumor killer cell.

24. The method of claim 21, wherein the tumor killer cell retains the ability to clear the tumor.

25. The method of claim 21, further comprising administering a chemotherapy to the subject.


SEQUENCE LISTING (88 sequences)

SEQ ID NO: 1 - RNA, 21 nt, synthetic construct
  source 1..21: mol_type = other RNA
ctcattaagaatgcttacaat

SEQ ID NO: 2 - RNA, 21 nt, synthetic construct
  source 1..21: mol_type = other RNA
atcccattaattatattgtta

SEQ ID NO: 3 - RNA, 21 nt, synthetic construct
  source 1..21: mol_type = other RNA
gtcccagtggaacaatgataa

SEQ ID NO: 4 - RNA, 21 nt, synthetic construct
  source 1..21: mol_type = other RNA
ggtgaagacctacgagaaact

SEQ ID NO: 5 - RNA, 21 nt, synthetic construct
  source 1..21: mol_type = other RNA
ggtgtgacactggctacattg

SEQ ID NO: 6 - RNA, 21 nt, synthetic construct
  source 1..21: mol_type = other RNA
gcattctcctgctggtcatct

SEQ ID NO: 7 - RNA, 21 nt, synthetic construct
  source 1..21: mol_type = other RNA
ggaccgctacctcatctatgt

SEQ ID NO: 8 - RNA, 21 nt, synthetic construct
  source 1..21: mol_type = other RNA
gaaacccaggaagaccacaat

SEQ ID NO: 9 - RNA, 22 nt, synthetic construct
  source 1..22: mol_type = other RNA
ctggtgaagacctacgagaaac

SEQ ID NO: 10 - RNA, 22 nt, synthetic construct
  source 1..22: mol_type = other RNA
ccaggagtgcacgaagttcaag

SEQ ID NO: 11 - RNA, 22 nt, synthetic construct
  source 1..22: mol_type = other RNA
aaggacatcagtctgattaaag

SEQ ID NO: 12 - RNA, 22 nt, synthetic construct
  source 1..22: mol_type = other RNA
cggtgcgtgtttcctgtgcaag

SEQ ID NO: 13 - RNA, 22 nt, synthetic construct
  source 1..22: mol_type = other RNA
ccccattaattatattgttaat

SEQ ID NO: 14 - RNA, 22 nt, synthetic construct
  source 1..22: mol_type = other RNA
gtgccaatttatttacatttaa

SEQ ID NO: 15 - RNA, 22 nt, synthetic construct
  source 1..22: mol_type = other RNA
tgccaatttatttacatttaaa

SEQ ID NO: 16 - RNA, 22 nt, synthetic construct
  source 1..22: mol_type = other RNA
cacgtgtatagaaaaaaaataa

SEQ ID NO: 17 - RNA, 22 nt, synthetic construct
  source 1..22: mol_type = other RNA
cacgacggtcatgaaccccaag

SEQ ID NO: 18 - RNA, 22 nt, synthetic construct
  source 1..22: mol_type = other RNA
cagggtcttcctggatcacaac

SEQ ID NO: 19 - RNA, 22 nt, synthetic construct
  source 1..22: mol_type = other RNA
ctgcgagtgtgacaccatcaac

SEQ ID NO: 20 - RNA, 22 nt, synthetic construct
  source 1..22: mol_type = other RNA
tagaaaaaaaataaaacttcaa

SEQ ID NO: 21 - RNA, 22 nt, synthetic construct
  source 1..22: mol_type = other RNA
cccgatcaccttccaggtgaag

SEQ ID NO: 22 - RNA, 21 nt, synthetic construct
  source 1..21: mol_type = other RNA
gagcgagtttgtgaaaattct

SEQ ID NO: 23 - RNA, 21 nt, synthetic construct
  source 1..21: mol_type = other RNA
aaccagtaacaagatgaaaga

SEQ ID NO: 24 - RNA, 21 nt, synthetic construct
  source 1..21: mol_type = other RNA
gtgaggcaaacttgagagtgt

SEQ ID NO: 25 - RNA, 21 nt, synthetic construct
  source 1..21: mol_type = other RNA
gcacgccaatgtgacctgtaa

SEQ ID NO: 26 - RNA, 21 nt, synthetic construct
  source 1..21: mol_type = other RNA
ccaatctgtttccagatcaag

SEQ ID NO: 27 - RNA, 22 nt, synthetic construct
  source 1..22: mol_type = other RNA
atgcatgtatttatccaataaa

SEQ ID NO: 28 - RNA, 22 nt, synthetic construct
  source 1..22: mol_type = other RNA
acacatgttgctgttgaccaat

SEQ ID NO: 29 - RNA, 22 nt, synthetic construct
  source 1..22: mol_type = other RNA
cagcagctatctctcagtgaac

SEQ ID NO: 30 - RNA, 22 nt, synthetic construct
  source 1..22: mol_type = other RNA
cggaagagaatgtctgatctaa

SEQ ID NO: 31 - RNA, 21 nt, synthetic construct
  source 1..21: mol_type = other RNA
accaaaagaggtctgttcata

SEQ ID NO: 32 - RNA, 21 nt, synthetic construct
  source 1..21: mol_type = other RNA
gagacaggccaccgaatatga

SEQ ID NO: 33 - RNA, 21 nt, synthetic construct
  source 1..21: mol_type = other RNA
ggagatacagaccactcaacc

SEQ ID NO: 34 - RNA, 21 nt, synthetic construct
  source 1..21: mol_type = other RNA
ggaagcacagaccactcaacc

SEQ ID NO: 35 - RNA, 22 nt, synthetic construct
  source 1..22: mol_type = other RNA
ctggagatacagaccactcaac

SEQ ID NO: 36 - RNA, 22 nt, synthetic construct
  source 1..22: mol_type = other RNA
ccggaccaatatccctctaaac

SEQ ID NO: 37 - RNA, 22 nt, synthetic construct
  source 1..22: mol_type = other RNA
ccggaagcacagaccactcaac

SEQ ID NO: 38 - RNA, 22 nt, synthetic construct
  source 1..22: mol_type = other RNA
cacgatagcggaatccttcaag

SEQ ID NO: 39 - RNA, 21 nt, synthetic construct
  source 1..21: mol_type = other RNA
gcaagtacagagctaggacat

SEQ ID NO: 40 - RNA, 21 nt, synthetic construct
  source 1..21: mol_type = other RNA
gctccgtgttatcaagattat

SEQ ID NO: 41 - RNA, 21 nt, synthetic construct
  source 1..21: mol_type = other RNA
cgggagcagtaatgaatgcaa

SEQ ID NO: 42 - RNA, 22 nt, synthetic construct
  source 1..22: mol_type = other RNA
aagagtgtttgtgtacatcaac

SEQ ID NO: 43 - RNA, 22 nt, synthetic construct
  source 1..22: mol_type = other RNA
ttggagttatatcaacagtaaa

SEQ ID NO: 44 - RNA, 22 nt, synthetic construct
  source 1..22: mol_type = other RNA
tggatgcaatctgtaaagaaaa

SEQ ID NO: 45 - RNA, 22 nt, synthetic construct
  source 1..22: mol_type = other RNA
acgactctacatgtcaaactac

SEQ ID NO: 46 - RNA, 22 nt, synthetic construct
  source 1..22: mol_type = other RNA
tacaatataactacaaataaat

SEQ ID NO: 47 - RNA, 22 nt, synthetic construct
  source 1..22: mol_type = other RNA
tggagaagtcttagacttgaaa

SEQ ID NO: 48 - RNA, 21 nt, synthetic construct
  source 1..21: mol_type = other RNA
ccaaatcatgtggagaatgta

SEQ ID NO: 49 - RNA, 21 nt, synthetic construct
  source 1..21: mol_type = other RNA
tttgtaggaagagggataata

SEQ ID NO: 50 - RNA, 21 nt, synthetic construct
  source 1..21: mol_type = other RNA
gccctccagatgacatagaaa

SEQ ID NO: 51 - RNA, 21 nt, synthetic construct
  source 1..21: mol_type = other RNA
gccttgcattactgctgatat

SEQ ID NO: 52 - RNA, 21 nt, synthetic construct
  source 1..21: mol_type = other RNA
tcccacaacactgaatgcaaa

SEQ ID NO: 53 - RNA, 21 nt, synthetic construct
  source 1..21: mol_type = other RNA
agtgtggtgtctgtaagtgta

SEQ ID NO: 54 - RNA, 21 nt, synthetic construct
  source 1..21: mol_type = other RNA
tgtcttgactctgatgtattt

SEQ ID NO: 55 - RNA, 21 nt, synthetic construct
  source 1..21: mol_type = other RNA
gagcaagagagccggataaat

SEQ ID NO: 56 - RNA, 21 nt, synthetic construct
  source 1..21: mol_type = other RNA
gattgacaaatacgatttatc

SEQ ID NO: 57 - RNA, 20 nt, synthetic construct
  source 1..20: mol_type = other RNA
atttagacatttttacagga

SEQ ID NO: 58 - RNA, 20 nt, synthetic construct
  source 1..20: mol_type = other RNA
tcatcacatcgtgcagaagt

SEQ ID NO: 59 - RNA, 20 nt, synthetic construct
  source 1..20: mol_type = other RNA
tttagaagccttaaaaaaga

SEQ ID NO: 60 - RNA, 20 nt, synthetic construct
  source 1..20: mol_type = other RNA
ttagaagccttaaaaaagaa

SEQ ID NO: 61 - RNA, 20 nt, synthetic construct
  source 1..20: mol_type = other RNA
gtctcgctttaggctcctag

SEQ ID NO: 62 - RNA, 20 nt, synthetic construct
  source 1..20: mol_type = other RNA
tctcgctttaggctcctagt

SEQ ID NO: 63 - RNA, 20 nt, synthetic construct
  source 1..20: mol_type = other RNA
tggcagtgggcgcacccact

SEQ ID NO: 64 - RNA, 20 nt, synthetic construct
  source 1..20: mol_type = other RNA
ttggcgagccagttggcagt

SEQ ID NO: 65 - RNA, 20 nt, synthetic construct
  source 1..20: mol_type = other RNA
gcactaccagagctaactca

SEQ ID NO: 66 - RNA, 20 nt, synthetic construct
  source 1..20: mol_type = other RNA
cgttcaacgtgaccttccgg

SEQ ID NO: 67 - RNA, 20 nt, synthetic construct
  source 1..20: mol_type = other RNA
gccgggaatgcatcgagtcg

SEQ ID NO: 68 - RNA, 20 nt, synthetic construct
  source 1..20: mol_type = other RNA
tcagatagtacaggtcgatg

SEQ ID NO: 69 - RNA, 20 nt, synthetic construct
  source 1..20: mol_type = other RNA
ccagaacttcacagggccgg

SEQ ID NO: 70 - RNA, 20 nt, synthetic construct
  source 1..20: mol_type = other RNA
cacgtccaggttgtagctcg

SEQ ID NO: 71 - RNA, 20 nt, synthetic construct
  source 1..20: mol_type = other RNA
gccggcctcgagctacaacc

SEQ ID NO: 72 - RNA, 20 nt, synthetic construct
  source 1..20: mol_type = other RNA
cacacgttcgagacagccca

SEQ ID NO: 73 - RNA, 20 nt, synthetic construct
  source 1..20: mol_type = other RNA
gtagctcgaggccggcgctg

SEQ ID NO: 74 - RNA, 20 nt, synthetic construct
  source 1..20: mol_type = other RNA
atctaggtactcatattcgg

SEQ ID NO: 75 - RNA, 20 nt, synthetic construct
  source 1..20: mol_type = other RNA
caggaggagttgcagaggca

SEQ ID NO: 76 - RNA, 20 nt, synthetic construct
  source 1..20: mol_type = other RNA
cactcaaccagtgcccacgg

SEQ ID NO: 77 - RNA, 20 nt, synthetic construct
  source 1..20: mol_type = other RNA
gcttgcccgggaccggagac

SEQ ID NO: 78 - DNA, 1455 bp, synthetic construct
  source 1..1455: mol_type = other DNA
atgcttctcctggtgacaagccttctgctctgtgagttaccacacccagcattcctcctgatcccagaggtgcagctggccgagagcggcgggggcctggtgcagcccggcagaagcatgaagctgagctgcgccgctagcggcttcaccttcagcaacttccccatggcctgggtgagacaagcccccaccaagggcctggagtgggtggccaccatcagcacaagcggcggcagcacctactacagagacagcgtgaagggcagattcaccatcagcagagacaacgccaagagcaccctgtacctgcagatgaacagcctgagaagcgaggacaccgccacctactactgcacaagaaccctgtacatcctgagagtgttctacttcgactactggggccaaggcgtgatggtgaccgtgagcagcggcgggggcggcagcggcggcgggggcagcggcggcgggggcagcgacattcagatgacacagagccccgctagcctgagcgctagcctgggcgagaccgtgagcatcgagtgcctggctagcgagggcatcagcaacgacctggcctggtatcagcagaagagcggcaagagccctcagctgctgatctacgccacaagcagactgcaagacggcgtgcctagcagattcagcggcagcggcagcggcacaagatacagcctgaagatcagcggcatgcagcccgaggacgaggccgactacttctgtcagcagagctacaagtacccctggaccttcggcgggggcaccaagctggagctgaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 79 - DNA, 1473 bp, synthetic construct
  source 1..1473: mol_type = other DNA
atgcttctcctggtgacaagccttctgctctgtgagttaccacacccagcattcctcctgatcccacaggttcagctggttcagtctggcgccgaagtgaagaaacctggcgcctctgtgaaggtgtcctgcaaggcttctggctacacctttaccaactactggatcaactgggtccgacaggctcctggacagggactcgagtggatcggcaacatctaccccagctacatctacaccaattacaatcaagagttcaaggacaaagtgaccctgaccgtggacgagtccacctccaccgcttacatggaactgtccagcctgagatccgaggacaccgccgtgtactactgcaccagatctccctacggctacgacgagtacggcctggattattggggccagggcaccacagtgaccgtgtcatctggtggaggcggttcaggcggaggtggctctggcggtggcggatcggacatccagctgacccagtctccatcctctctgtctgcctctgtgggcgacagagtgaccatgacctgcaagtcctctcagtccctgctgaacacccggaaccagaagaactacctgacctggtatcagcagaagcccggcaaggctcccaagctgctgatctactgggcctccaccagagaatctggcgtgccctctagattctccggctctggctctggcaccgactttaccctgacaatctccagcctgcagcctgaggacttcgccacctactactgccagaacgactacgtgtaccctctgacctttggccagggcaccaagctggaaatcaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 80 - DNA, 1470 bp, synthetic construct
  source 1..1470: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 81 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 82 - DNA, 81 bp, synthetic construct
  source 1..81: mol_type = other DNA
ttttgggtgctggtggtggttgggggagtcctggcttgctatagcttgctagtaacagtggcctttattattttctgggtg

SEQ ID NO: 83 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 84 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 85 - RNA, 396 nt, synthetic construct
  source 1..396: mol_type = other RNA
  modified_base 295..315: mod_base = OTHER; note = The nnnnnnnnnnnnnnnnnnnnn denotes the insertion sites of the seed sequence of shRNA and its complementary sequence
  modified_base 322..342: mod_base = OTHER; note = The nnnnnnnnnnnnnnnnnnnnn denotes the insertion sites of the seed sequence of shRNA and its complementary sequence
actagtatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggnnnnnnnnnnnnnnnnnnnnnctcgagnnnnnnnnnnnnnnnnnnnnntttttgaattctcgacctcgagacaaatggcagtattcatccacaaggcgcgcc

SEQ ID NO: 86 - RNA, 80 nt, synthetic construct
  source 1..80: mol_type = other RNA
  modified_base 77^78: mod_base = OTHER; note = Phosphorothioate Linkage
  modified_base 78^79: mod_base = OTHER; note = Phosphorothioate Linkage
  modified_base 79^80: mod_base = OTHER; note = Phosphorothioate Linkage
  modified_base 78..80: mod_base = m5u
gttttagagctagaaatagcaagttaaaataaggctagtccgttatcaacttgaaaaagtggcaccgagtcggtgctttt

SEQ ID NO: 87 - DNA, 57 bp, synthetic construct
  source 1..57: mol_type = other DNA
ccggnnnnnnnnnnnnnnnnnnnnnctcgagnnnnnnnnnnnnnnnnnnnnnttttt

SEQ ID NO: 88 - DNA, 57 bp, synthetic construct
  source 1..57: mol_type = other DNA
aaaaannnnnnnnnnnnnnnnnnnnnctcgagnnnnnnnnnnnnnnnnnnnnnccgg